(19) United States
(12) Patent Application Publication
(10) Pub. No.: US 2017/0226535 A1
(43) Pub. Date: Aug. 10, 2017

(54) Specialized Activated Carbon Derived From Pretreated Biomass

(71) Applicant: Sweetwater Energy, Inc., Rochester, NY (US)

(72) Inventor: Scott TUDMAN, Rochester, NY (US)

(21) Appl. No.: 15/267,617

(22) Filed: Sep. 16, 2016

Related U.S. Application Data
(60) Provisional application No. 62/219,476, filed on Sep. 16, 2015.

(51) Int. Cl. C12P 3/00; C12M 1/34; C12M 1/00; C12M 1/40

(52) U.S. Cl. CPC C12P 3/00; C12M 21/18; C12M 41/12; C12M 47/10

(57) ABSTRACT

Provided are methods, systems, and compositions for producing activated carbon from lignin residues produced from cellulosic or lignocellulosic biomass after hydrolysis of saccharides. The activated carbon is low in ash and sulfur, high in oxygen content and iodine number.

### CROSS-REFERENCE

This application claims the benefit of U.S. Provisional Application No. 62/219,476, filed Sep. 16, 2015, which application is incorporated herein by reference in its entirety.

### BACKGROUND OF THE INVENTION

Currently most of the global supply for fermentable refined C6 sugars is derived by processing renewable feedstocks rich in starch. The lignin-rich residues (lignin material) remaining after this process is a product that, to date, has found few economical uses. Activated carbon, also called activated charcoal or activated coal, is a charcoal product with a micropore structure that exhibits a significant specific internal surface area through its porosity. It has many uses, including the adsorption of unwanted materials. The present disclosure addresses an unmet need in the art and relates to the production of specialized activated carbon from lignocellulosic residues.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a block diagram depicting one of several stage pretreatment processes, showing the biomass feedstock entering into the pretreatement and hydrolysis process system, thereby producing sugar hydrolysate products (sugar stream) and activated carbon from a lignin residue solids product.

FIG. 2 compares the pore volume of different activated carbons compared to sample EE-634A2.

FIG. 3 is a graph depicting the raw characterization data of various activated carbons compared to sample EE-634A2.

FIGS. 4a and 4b are graphs depicting volume-based differential characteristic curves of various activated carbons compared to sample EE-634A2.

FIG. 5 is a graph depicting the adsorption isotherm for MTBE of various activated carbons compared to sample EE-634A2.

FIGS. 6a and 6b are graphs depicting the adsorption isotherms for Benzene and Phenol, respectively, of various activated carbons compared to sample EE-634A2.

FIG. 7 is a graph depicting the pore size distribution of various activated carbons compared to sample EE-634A2.

### DETAILED DESCRIPTION OF THE INVENTION

As used in the specification and the appended claims, the singular forms “a,” “an” and “the” include plural referents unless the context clearly dictates otherwise. Thus, for example, reference to “a purified monomer” includes mixtures of two or more purified monomers. The term “comprising” as used herein is synonymous with “including,” “containing,” or “characterized by,” and is inclusive or open-ended and does not exclude additional, unrecited elements or method steps.

“About” means a referenced numeric indication plus or minus 10% of that referenced numeric indication. For example, the term about 4 would include a range of 3.6 to 4.4. All numbers expressing quantities of ingredients, reaction conditions, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Wherever the phrase “for example,” “such as,” “including” and the like are used herein, the phrase “and without limitation” is understood to follow unless explicitly stated otherwise. Therefore, “for example ethanol production” means “for example and without limitation ethanol production.”

### INTRODUCTION

Activated carbon, also called activated charcoal or activated coal, is a charcoal product with a micropore structure that exhibits a significant specific internal surface area through its porosity. It has many uses, including the adsorption of unwanted materials. Thus, it can be used for water purification, sewage treatment, gas purification, decaffeination, gold purification, air filters in gas masks and respirators, filters in compressed air, metal extraction, color removal, medicinal uses, absorption of nitrogen for slow release fertilizer, sound absorption, and many other applications. Because activated carbon has so many uses, additional types of the product and methods of its manufacture would be beneficial to improve these applications.

The surface area of one gram of activated carbon is typically about 500 m² and ranges from about 200 m² to about 2500 m². Physically, activated carbon binds materials by van der Waals force or London dispersion force. Iodine is adsorbed especially well and its iodine adsorption capacity is used as a standard indication of total surface area and activity level. A higher mg/g level indicates a higher degree of activation. Iodine number is defined as the milligrams of iodine adsorbed by one gram of carbon when the iodine concentration in the residual filtrate is 0.02 normal.

Some residual substances in activated carbons can reduce its overall activity and its reactivation potential. One such substance is ash. The ash levels in activated carbon become especially important when it is used in aqueous solutions to adsorb undesirable substances because metal oxides such as Fe₂O₃ can leach out of the ash-laden activated carbon causing discoloration, heavy metal toxicity, and excessive algal growth.

Activated carbon can be produced from carbon-containing (carbonaceous) materials such as coconut husk, wood, including chips, sawdust and bark, nutshells, agricultural residues, peat, coal, lignite, petroleum pitch, and the like. First, the pure carbon can be extracted by a heating method, usually pyrolysis. Then, once the material is carbonized, it can be activated, or treated with oxygen, either by exposure to CO₂ or steam, or by an acid-base chemical treatment.

For carbonization, carbon-rich material can be placed in a small (relative to the amount of material) furnace and cooked at extreme temperatures up to 2000 degrees Celsius. What remains is usually 20-30 percent of the beginning weight, and consists of mostly carbon and a small percentage of inorganic ash. This is very similar to “coking,” a method of producing coke from charcoal, a type of carbon-based fuel.

Activation can be done, for example, by one of two ways: gasification or chemical treatment. Activation by gasification involves directly heating the carbon in a chamber while gas is pumped in to oxygenate the carbon. Oxidation makes the carbon susceptible to adsorption, surface bonding for chemicals. Prolysis takes place in an inert environment at 600-900° C. Then, an oxygenated gas is pumped in while heating to between 900 and 1200° C., causing the oxygen to bond to the carbon's surface. In chemical treatment, the process is slightly different. For one, carbonization and chemical activation occur simultaneously. A bath of acid, base or other chemicals is prepared and the material submerged. The bath is then heated to temperatures of 450-900° C. much less than the heat needed for gas activation. The carbonaceous material is carbonized and then activated, all at a much quicker pace than gas activation. However, some heating processes cause trace elements from the bath to adsorb to the carbon, which can result in impure or ineffective active carbon.

Following oxidization, activated carbon can be processed for many different kinds of uses, with several classifiably different properties. Some of these classes are powdered activated carbon (PAC), granular activated carbon (GAC), extruded activated carbon (EAC), and bead activated carbon (BAC).

Lignin residues are a common by-product of sugar extraction from biomass. The sugars can then be used to produce other energy-rich products such as ethanol, other fuels, bioplastics and the like. The lignin residues vary considerably depending on the type of process and equipment used to hydrolyze and extract the sugar. In many instances, these residues can be used for production of activated carbon, and are especially suited for particular adsorption applications.

Activated carbon is a form of carbon that has been altered, derivatized, or modified or so called “activated” to further improve its physiochemical properties for various industrial applications. Activated carbon is typically reduced in particle size and its surface is covered in low volume pores which increases the surface area for absorption. There are many types of activated carbon used in industry and, depending on the processing methods used in their manufacture, these serve various purposes. Activated carbon can be produced from raw materials such as anthracite or bituminous coal as well as from raw vegetable or woody materials, such as coconut shells, wood chips, and the like, that are rich in carbon but also contain sugars, proteins, fats, oils, and other compounds. See, e.g., U.S. Pat. No. 8,926,932 B2. Also, some of the woody feedstock used can be from pulp and paper industrial by-products made through the Kraft process, and other processes that result in a lignin-rich residue but one that can be highly-sulfonated and wherein the reactive sites on the lignin molecules are blocked. Activated carbon can also be made from animal matter such as bones, restaurant and other food waste, and carcasses.

Further, all of these types of processes, whether the lignin feedstock is the whole or partial plant, or produced by an extraction process through chemical pulping process such as the black liquor from the Kraft process, or steam-explosion, high-temperature pyrolysis, or another method, can result in long carbon fibers and a high ash content, and often, as in the case of pyrolysis, a condensed material with reduced pores. See, e.g., U.S. Publication 2015/0197424 A1. The activated carbon produced by these processes is not nearly as readily reactive as an activated carbon with many small pores, low ash and low sulfur and considerable oxygen content. The processes described herein result in a more highly-porous, uniform pored, activated carbon that has an abundance of high energy pores with low ash, high oxygen and low sulfur content. The acid hydrolysis process used can be much faster and more effective than traditional pretreatment processes, and further processing steps can remove other impurities such as enzymes, acids, sugars and other residues, yielding a refined lignin prior to carbonization and activation. These sugars can be used to make useful end-products such as biofuels and bioplastics. Further, the homogenous and consistently small particle size of the starting material (ensuring the lignin residues have a small particle size), are derived through the removal of the cellulose and hemicellulose.

Currently most of the global supply for fermentable refined C6 sugars is derived by processing renewable feedstocks rich in starch, such as corn, rice, cassava, wheat, sorghum and in few cases, cane sugar (comprised of glucose and fructose). Production of refined C6 sugars from these feedstocks is well established and is relatively simple because the starch is concentrated in particular plant parts (mostly seeds) and can be easily isolated and hydrolyzed to monomeric sugars using amylase enzymes. Saccharification is performed at low temperatures, resulting in fewer inhibitors and breakdown products. Starch is typically a white amorous powder and does not contain any interfering complex phenolics, acids, extractives, or colored compounds. Even if these are present, they are in such low quantity that, it is easy to refine and remove these compounds. These attributes have enabled corn refiners and starch processing companies then to provide highly-concentrated, refined sugars within tight specifications at low cost using anion exchange columns and low levels of sequestering agents. However, the remaining lignin-rich residues (lignin material) remaining after separation of most of the sugar streams is a product that, to date, has found few economical uses. For the most part, it is burned as an energy source to produce the heat and pressure necessary to pretreat biomass, or as a feedstock for cattle and other livestock.

Lignocellulosic biomass, including wood, can require high temperatures to depolymerize the sugars contained within and, in some cases, explosion and more violent reaction with steam (explosion) and/or acid to make the biomass ready for enzyme hydrolysis. The C5 and C6 sugars are naturally embedded in and cross-linked with lignin, extractives and phenolics. The high temperature and pressures can result in the leaching of lignin and aromatics, loading with mixed sugars, high ash, lignin aromatic fragments, inhibitors, and acids in stream. Further enzymatic hydrolysis converts most of the sugars to product valuable feedstock that can be further processed to ethanol or another alcohol, and a variety of other biochemical and bioproducts. After enzymatic hydrolysis, the lignin can be separated from the sugar product. Separation of the lignin residues can be accomplished via flocculation, filtration, and/or centrifugation, or other methods. The extracted lignin residues can have a very porous structure, and can contain small amounts of ash, enzymes, sulfur, sugars, and other products. The resulting lignin-rich product chars at lower temperatures than typical carbon feedstocks, and when it is carbonized and activated, it forms an activated carbon that is especially suited to specialized uses such as removing organic compounds from drinking water. In fact, depending on the processing conditions, raw lignin-derived activated carbon performs as well or better than coconut shell activated carbon for organics removal from water. Further, and without being bound by theory, it appears that the low residual sugar combined with the small particle size lignin contribute to a smaller and more uniform pore size resulting in a higher surface area activated carbon that has a large percentage of small, high energy pore sizes that are well suited for organics adsorption and other applications.

In this specification and in the claims that follow, reference will be made to a number of terms which shall be defined to have the following meanings.

### Definitions

“Optional” or “optionally” means that the subsequently described event or circumstance may or may not occur, and that the description includes instances where said event or circumstance occurs and instances where it does not. For example, the phrase “the medium can optionally contain glucose” means that the medium may or may not contain glucose as an ingredient and that the description includes both media containing glucose and media not containing glucose.

Unless characterized otherwise, technical and scientific terms used herein have the same meaning as commonly understood by one of ordinary skill in the art.

“Fermentive end-product” and “fermentation end-product” are used interchangeably herein to include biofuels, chemicals, compounds suitable as liquid fuels, gaseous fuels, triacylglycerols, reagents, chemical feedstocks, chemical additives, processing aids, food additives, bioplastics and precursors to bioplastics, and other products.

Fermentation end-products can include polyols or sugar alcohols; for example, methanol, glycol, glycerol, erythritol, threitol, arabitol, xylitol, ribitol, mannitol, sorbitol, dulcitol, fucitol, iditol, inositol, volemitol, isomalt, maltitol, lactitol, and/or polyglycitol.

The term “fatty acid comprising material” as used herein has its ordinary meaning as known to those skilled in the art and can comprise one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc.

The term “pH modifier” as used herein has its ordinary meaning as known to those skilled in the art and can include any material that will tend to increase, decrease or hold steady the pH of the broth or medium. A pH modifier can be an acid, a base, a buffer, or a material that reacts with other materials present to serve to raise, lower, or hold steady the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. In one embodiment, a buffer can be produced in the broth or medium or separately and used as an ingredient by at least partially reacting in acid or base with a base or an acid, respectively. When more than one pH modifiers are utilized, they can be added at the same time or at different times. In one embodiment, one or more acids and one or more bases are combined, resulting in a buffer. In one embodiment, media components, such as a carbon source or a nitrogen source serve as a pH modifier; suitable media components include those with high or low pH or those with buffering capacity. Exemplary media components include acid- or base-hydrolyzed plant polysaccharides having residual acid or base, ammonia fiber explosion (AFEX) treated plant material with residual ammonia, lactic acid, corn steep solids or liquor.

The term “lignin” as used herein has its ordinary meaning as known to those skilled in the art and can comprise a cross-linked organic, racemic phenol polymer with molecular masses in excess of 10,000 microns that is relatively hydrophobic and aromatic in nature. Its degree of polymerization in nature is difficult to measure, since it is fragmented during extraction and the molecule consists of various types of substructures that appear to repeat in a haphazard manner. There are three monolignol monomers, methoxylated to various degrees: p-coumaryl alcohol, coniferyl alcohol, and sinapyl alcohol. These lignols are incorporated into lignin in the form of the phenylpropanoids p-hydroxyphenyl (H), guaiacyl (G), and syringyl (S), respectively. All lignins contain small amounts of incomplete or modified monolignols, and other monomers are prominent in non-woody plants. Lignins are one of the main classes of structural materials in the support tissues of vascular and nonvascular plants and some algae. Lignins are particularly important in the formation of cell walls, especially in wood and bark.

The term “pyrolysis” as used herein has its ordinary meaning as known to those skilled in the art and generally refers to thermal decomposition of a carbonaceous material. In pyrolysis, less oxygen is present than is required for complete combustion, such as less than 10%. In some embodiments, pyrolysis can be performed in the absence of oxygen.

The term “ash” as used herein has its ordinary meaning as known to those skilled in the art and generally refers to any solid residue that remains following a combustion process that is not volatilized and remains as solid residue, and is not limited in its composition. Ash is generally rich in metal oxides, such as SiO₂, CaO, Al₂O₃, and K₂O. “Carbon-containing ash” or “carbonized ash” means ash that has at least some carbon content. Fly ash, also known as flue ash, is one of the residues generated in combustion, and comprises the fine particles that rise with the flue gases. Ash which does not rise is termed bottom ash. Fly ash is generally captured by electrostatic precipitators or other particle filtration equipment before the flue gases are emitted. The bottom ash is typically removed from the bottom of the furnace.

The term “plant polysaccharide” as used herein has its ordinary meaning as known to those skilled in the art and can comprise one or more polymers of sugars and sugar derivatives as well as derivatives of sugar polymers and/or other polymeric materials that occur in plant matter. Exemplary plant polysaccharides include cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives.

The terms “SSF” and “SHF” are known to those skilled in the art; “SSF” meaning simultaneous saccharification and fermentation, or the conversion from polysaccharides or oligosaccharides into monosaccharides at the same time and in the same fermentation vessel wherein monosaccharides are converted to another chemical product such as ethanol. “SHF” indicates a physical separation of the polymer hydrolysis or saccharification and fermentation processes.

The term “biomass” as used herein has its ordinary meaning as known to those skilled in the art and can include one or more carbonaceous biological materials that can be converted into a biofuel, chemical or other product. Biomass as used herein is synonymous with the term “feedstock” and includes corn syrup, molasses, silage, agricultural residues (corn stalks, grass, straw, grain hulls, bagasse, etc.), nuts, nut shells, coconut shells, animal waste (manure from cattle, poultry, and hogs), Distillers Dried Solubles (DDS), Distillers Dried Grains (DDG), Condensed Distillers Solubles (CDS), Distillers Wet Grains (DWG), Distillers Dried Grains with Solubles (DDGS), woody materials (wood or bark, sawdust, wood chips, timber slash, and mill scrap), municipal waste (waste paper, recycled toilet papers, yard clippings, etc.), and energy crops (poplars, willows, switchgrass, alfalfa, prairie bluestem, algae, including macroalgae, etc.). One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, sorghum, high biomass sorghum, bamboo, algae and material derived from these. Plants can be in their natural state or genetically modified, e.g., to increase the cellulosic or hemicellulosic portion of the cell wall, or to produce additional exogenous or endogenous enzymes to increase the separation of cell wall components. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corncobs, corn fiber, corn steep solids, distiller's grains, peels, pits, fermentation waste, straw, lumber, sewage, garbage and food leftovers. Peels can be citrus which include, but are not limited to, tangerine peel, grapefruit peel, orange peel, tangerine peel, lime peel and lemon peel. These materials can come from farms, forestry, industrial sources, households, etc. Another non-limiting example of biomass is animal matter, including, for example milk, bones, meat, fat, animal processing waste, and animal waste. “Feedstock” is frequently used to refer to biomass being used for a process, such as those described herein.

“Concentration” when referring to material in the broth or in solution generally refers to the amount of a material present from all sources, whether made by the organism or added to the broth or solution. Concentration can refer to soluble species or insoluble species, and is referenced to either the liquid portion of the broth or the total volume of the broth, as for “titer.” When referring to a solution, such as “concentration of the sugar in solution”, the term indicates increasing one or more components of the solution through evaporation, filtering, extraction, etc., by removal or reduction of a liquid portion.

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art. For example, a biocatalyst can be a fermenting microorganism.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microbes, and can include the enzymatic hydrolysis of released carbohydrate polymers or oligomers to monomers. In one embodiment, pretreatment includes removal or disruption of lignin so as to make the cellulose and hemicellulose polymers in the plant biomass more available to cellulolytic enzymes and/or microbes, for example, by treatment with acid or base. In one embodiment, pretreatment includes disruption or expansion of cellulosic and/or hemicellulosic material. In another embodiment, it can refer to starch release and/or enzymatic hydrolysis to glucose. Steam explosion, and ammonia fiber expansion (or explosion) (AFEX) are well known thermal/chemical techniques. Hydrolysis, including methods that utilize acids, bases, and/or enzymes can be used. Other thermal, chemical, biochemical, enzymatic techniques can also be used.

“Sugar compounds” or “sugar streams” is used herein to indicate mostly monosaccharide sugars, dissolved, crystallized, evaporated, or partially dissolved, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length. A sugar stream can consist of primarily or substantially C6 sugars, C5 sugars, or mixtures of both C6 and C5 sugars in varying ratios of said sugars. C6 sugars have a six-carbon molecular backbone and C5 sugars have a five-carbon molecular backbone.

A “liquid” composition may contain solids and a “solids” composition may contain liquids. A liquid composition refers to a composition in which the material is primarily liquid, and a solids composition is one in which the material is primarily solid.

### DESCRIPTION

The following description and examples illustrate some exemplary embodiments of the disclosure in detail. Those of skill in the art will recognize that there are numerous variations and modifications of this disclosure that are encompassed by its scope. Accordingly, the description of a certain exemplary embodiment should not be deemed to limit the scope of the present disclosure.

In the extraction of carbohydrate from biomass, lignin has been an unwelcome byproduct, adding difficulty and expense to the separation of biomass components. The amount of lignin in plant materials varies widely. In wood, it ranges from approximately 12-39% of the dry weight.

Steam explosion and/or acid hydrolysis of lignocellulosic biomass to produce sugars can be costly and requires special equipment. The process, especially under high temperatures and pressure, can release structural carbohydrates in cellulosic biomass and can expose crystalline cellulose to enzymatic degradation. The byproducts of acid hydrolysis and subsequent enzymatic hydrolysis (SHF) is a solids mixture of unfermented carbohydrate, lignin, protein and minerals, often called “lignin residues.” On a dry weight basis, the carbohydrate portion can vary from 1-30%. The protein component ranges from 1-5% and minerals (ash) comprise from 1-4%. There will also be some remaining enzymes in the mixture. However, the largest component is lignin which ranges from 30-90%, depending on the type of biomass and the sugar separation and washing steps. This is also true of SSF processes which result in high lignin residues.

For the most part, the lignin residues are either fed to livestock or burned to produce energy.

Feedstock and Pretreatment of Feedstock

In one embodiment, the feedstock (biomass) contains cellulosic, hemicellulosic, and/or lignocellulosic material. The feedstock can be derived from agricultural crops, crop residues, trees, woodchips, sawdust, paper, cardboard, grasses, algae, municipal waste and other sources.

Cellulose is a linear polymer of glucose where the glucose units are connected via β(1→4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostly p-coumaryl alcohol, conferyl alcohol and sinapyl alcohol. These three polymers occur together in lignocellulosic materials in plant biomass. The different characteristics of the three polymers can make hydrolysis of the combination difficult as each polymer tends to shield the others from enzymatic attack.

In one embodiment, methods are provided for the pretreatment of feedstock for the release of sugars that can be used to further produce biofuels and biochemicals. The pretreatment steps can include mechanical, thermal, pressure, chemical, thermochemical, and/or biochemical treatment methods prior to being used in a bioprocess for the production of fuels and chemicals, but untreated biomass material can be used in the process as well. Mechanical processes can reduce the particle size of the biomass material so that it can be more conveniently handled in the bioprocess and can increase the surface area of the feedstock to facilitate contact with chemicals/biochemicals/biocatalysts. Mechanical processes can also separate one type of biomass material from another. The biomass material can also be subjected to thermal and/or chemical pretreatments to render plant polymers more accessible. Multiple steps of treatment can also be used.

Mechanical processes include, are not limited to, washing, soaking, milling, grinding, size reduction, screening, shearing, size classification and density classification processes. Chemical processes include, but are not limited to, bleaching, oxidation, reduction, acid treatment, base treatment, sulfite treatment, acid sulfite treatment, basic sulfite treatment, ammonia treatment, and hydrolysis. Thermal processes include, but are not limited to, sterilization, ammonia fiber expansion or explosion (“AFEX”), steam explosion, holding at elevated temperatures, pressurized or unpressurized, in the presence or absence of water, and freezing. Biochemical processes include, but are not limited to, treatment with enzymes, including enzymes produced by genetically-modified plants or organisms, and treatment with microorganisms. Various enzymes that can be utilized include cellulase, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal, thermochemical, and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a biomass material, such as cellulosic, hemicellulosic or lignocellulosic material. Examples include paper plants, cellulosics plants, distillation plants, cotton processing plants, and microcrystalline cellulose plants. The feedstock can also include cellulose-containing or cellulosic containing waste materials. The feedstock can also be biomass materials, such as wood, grasses, corn, starch, or sugar, produced or harvested as an intended feedstock for production of ethanol or other products such as by biocatalysts.

In another embodiment, a method can utilize a pretreatment process disclosed in U.S. Patents and Patent Applications US20040152881, US20040171136, US20040168960, US20080121359, US20060069244, US20060188980, US20080176301, 5693296, 6262313, US20060024801, 5969189, 6043392, US20020038058, U.S. Pat. No. 5,865,898, U.S. Pat. No. 5,865,898, U.S. Pat. Nos. 6,478,965, 5,986,133, or US20080280338, each of which is incorporated by reference herein in its entirety

In another embodiment, the AFEX process is used for pretreatment of biomass. In a preferred embodiment, the AFEX process is used in the preparation of cellulosic, hemicellulosic or lignocellulosic materials for fermentation to ethanol or other products. The process generally includes combining the feedstock with ammonia, heating under pressure, and suddenly releasing the pressure. Water can be present in various amounts. The AFEX process has been the subject of numerous patents and publications.

In another embodiment, the pretreatment of biomass comprises the addition of calcium hydroxide to a biomass to render the biomass susceptible to degradation. Pretreatment comprises the addition of calcium hydroxide and water to the biomass to form a mixture, and maintaining the mixture at a relatively high temperature. Alternatively, an oxidizing agent, selected from the group consisting of oxygen and oxygen-containing gasses, can be added under pressure to the mixture. Examples of carbon hydroxide treatments are disclosed in U.S. Pat. No. 5,865,898 to Holtzapple and S. Kim and M. T. Holzapple, Bioresource Technology, 96, (2005) 1994, incorporated by reference herein in its entirety.

In one embodiment, pretreatment of biomass comprises dilute acid hydrolysis. Example of dilute acid hydrolysis treatment are disclosed in T. A. Lloyd and C. E Wyman, Bioresource Technology, (2005) 96, 1967, incorporated by reference herein in its entirety.

In another embodiment, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety.

In one embodiment, pretreatment of biomass comprises aqueous ammonia recycle process (ARP). Examples of aqueous ammonia recycle process are described in T. H. Kim and Y. Y. Lee, Bioresource Technology, (2005) 96, 2007, incorporated by reference herein in its entirety.

In one embodiment, the above mentioned methods have two steps: a pretreatment step that leads to a wash stream, and an enzymatic hydrolysis step of pretreated-biomass that produces a hydrolysate stream. In the above methods, the pH at which the pretreatment step is carried out includes acid hydrolysis, hot water pretreatment, steam explosion or alkaline reagent based methods (AFEX, ARP, and lime pretreatments). Dilute acid and hot water treatment methods solubilize mostly hemicellulose, whereas methods employing alkaline reagents remove most lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual biomass leads to mixed sugars (C5 and C6) in the alkali based pretreatment methods, while glucose is the major product in the hydrolyzate from the low and neutral pH methods. In one embodiment, the treated material is additionally treated with catalase or another similar chemical, chelating agents, surfactants, and other compounds to remove impurities or toxic chemicals or further release polysaccharides.

In one embodiment, pretreatment of biomass comprises ionic liquid (IL) pretreatment. Biomass can be pretreated by incubation with an ionic liquid, followed by IL extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

In another embodiment, a method can utilize a pretreatment process disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens, U.S. Pat. No. 6,106,888 to Dale, et al., U.S. Pat. No. 6,176,176 to Dale, et al., PCT publication WO2008/020901 to Dale, et al., Felix, A., et al., Anim. Prod. 51, 47-61 (1990), Wais, A. C., Jr., et al., Journal of Animal Science, 35, No. 1,109-112 (1972), which are incorporated herein by reference in their entireties.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 1, 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In one embodiment, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In one embodiment, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, 3.0, 2.0, 2.5, 1.0 or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pretreated at an elevated temperature and/or pressure. In one embodiment biomass is pretreated at a temperature range of 20° C. to 400° C. In another embodiment biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment steam can be injected into a biomass containing vessel. In another embodiment the steam, hot water, or hot gas can be injected into a vessel jacket such that it heats, but does not directly contact the biomass.

In another embodiment, a biomass can be treated at an elevated pressure. In one embodiment biomass is pretreated at a pressure range of about ipsi to about 30 psi. In another embodiment biomass is pretreated at a pressure or about 50 psi, 100 psi, 150 psi, 200 psi, 250 psi, 300 psi, 350 psi, 400 psi, 450 psi, 500 psi, 550 psi, 600 psi, 650 psi, 700 psi, 750 psi, 800 psi or more up to 900 psi. In some embodiments, biomass can be treated with elevated pressures by the injection of steam into a biomass containing vessel. In one embodiment, the biomass can be treated to vacuum conditions prior or subsequent to alkaline or acid treatment or any other treatment methods provided herein.

In one embodiment alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In one embodiment of the present invention, a pretreatment step includes a step of solids recovery. The solids recovery step can be during or after pretreatment (e.g., acid or alkali pretreatment), or before the drying step. In one embodiment, the solids recovery step provided by the methods of the present invention includes the use of flocculation, centrifugation, a sieve, filter, screen, or a membrane for separating the liquid and solids fractions. In one embodiment a suitable sieve pore diameter size ranges from about 0.001 microns to 8 mm, such as about 0.005 microns to 3 mm or about 0.01 microns to 1 mm. In one embodiment a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more. In one embodiment, biomass (e.g. corn stover) is processed or pretreated prior to fermentation. In one embodiment a method of pre-treatment includes but is not limited to, biomass particle size reduction, such as for example shredding, milling, chipping, crushing, grinding, or pulverizing. In one embodiment, biomass particle size reduction can include size separation methods such as sieving, or other suitable methods known in the art to separate materials based on size. In one embodiment size separation can provide for enhanced yields. In one embodiment, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In one embodiment, NaOH is added at a concentration of about 0.5% to about 2% by weight of the feedstock. In one embodiment, pretreatment also comprises addition of a chelating agent.

Hydrolysis

In one embodiment, the biomass hydrolyzing unit provides useful advantages for the conversion of biomass to biofuels and chemical products. One advantage of this unit is its ability to produce monomeric sugars, or monomeric and oligomeric sugars from multiple types of biomass, including mixtures of different biomass materials, and is capable of hydrolyzing polysaccharides and higher molecular weight saccharides to lower molecular weight saccharides. In one embodiment, the hydrolyzing unit utilizes a pretreatment process and a hydrolytic enzyme which facilitates the production of a sugar stream containing a concentration of a monomeric or monomeric and oligomeric sugars or several monomeric sugars, or monomeric and oligomeric sugars derived from cellulosic and/or hemicellulosic polymers. Examples of biomass material that can be pretreated and hydrolyzed to manufacture sugar monomers or monomers and oligomers include, but are not limited to, cellulosic, hemicellulosic, lignocellulosic materials; pectins; starches; wood; paper; agricultural products; forest waste; tree waste; tree bark; sawdust, wood chips, leaves; grasses; sawgrass; woody plant matter; non-woody plant matter; carbohydrates; starch; inulin; fructans; glucans; corn; corcobs, corn fiber, sugar cane; sorghum, other grasses; bamboo, algae, and material derived from these materials. This ability to use a very wide range of pretreatment methods and hydrolytic enzymes gives distinct advantages in biomass fermentations. Various pretreatment conditions and enzyme hydrolysis can enhance the extraction of sugars from biomass, resulting in higher yields, higher productivity, greater product selectivity, and/or greater conversion efficiency of the saccharides during fermentation and resulting in a more pure lignin residue.

In one embodiment, the enzyme treatment is used to hydrolyze various higher saccharides (higher molecular weight) present in biomass to lower saccharides (lower molecular weight), such as in preparation for fermentation by biocatalysts such as yeasts to produce ethanol, hydrogen, or other chemicals such as organic acids including succinic acid, formic acid, acetic acid, and lactic acid. These enzymes and/or the hydrolysate can be used in fermentations to produce various products including fuels, and other chemicals.

In one example, the process for converting biomass material into ethanol includes pretreating the biomass material (e.g., “feedstock”), hydrolyzing the pretreated biomass to convert polysaccharides to oligosaccharides, further hydrolyzing the oligosaccharides to monosaccharides, and converting the monosaccharides to biofuels and chemical products. Enzymes such as cellulases, polysaccharases, lipases, proteases, ligninases, and hemicellulases, help produce the monosaccharides can be used in the biosynthesis of fermentation end-products. Biomass material that can be utilized includes woody plant matter, non-woody plant matter, sawdust, wood chips, cellulosic material, lignocellulosic material, hemicellulosic material, carbohydrates, pectin, starch, inulin, fructans, glucans, corn, corn fiber, algae, sugarcane, other grasses, switchgrass, bagasse, wheat straw, barley straw, rice straw, corncobs, bamboo, citrus peels, sorghum, high biomass sorghum, seed hulls, nuts, nut shells, and material derived from these. The final product can then be separated and/or purified, as indicated by the properties for the desired final product. In some instances, compounds related to sugars such as sugar alcohols or sugar acids can be utilized as well.

Chemicals used in the methods of the present invention are readily available and can be purchased from a commercial supplier, such as Sigma-Aldrich. Additionally, commercial enzyme cocktails (e.g. Accellerase™ 1000, CelluSeb-TL, CelluSeb-TS, Cellic™ CTec, STARGEN™, Maxalign™, Spezyme. R™, Distillase. R™, G-Zyme. R™, Fermenzyme. R™, Fermgen™, GC 212, or Optimash™) or any other commercial enzyme cocktail can be purchased from vendors such as Specialty Enzymes & Biochemicals Co., Genencor, or Novozymes. Alternatively, enzyme cocktails can be prepared by growing one or more organisms such as for example a fungi (e.g. a Trichoderma, a Saccharomyces, a Pichia, a White Rot Fungus etc.), a bacteria (e.g. a Clostridium, or a coliform bacterium, a Zymomonas bacterium, Sacharophagus degradans etc.) in a suitable medium and harvesting enzymes produced therefrom. In some embodiments, the harvesting can include one or more steps of purification of enzymes.

In one embodiment, treatment of biomass comprises enzyme hydrolysis. In one embodiment a biomass is treated with an enzyme or a mixture of enzymes, e.g., endonucleases, exonucleases, cellobiohydrolases, cellulase, beta-glucosidases, glycoside hydrolases, glycosyltransferases, lyases, esterases and proteins containing carbohydrate-binding modules. In one embodiment, the enzyme or mixture of enzymes is one or more individual enzymes with distinct activities. In another embodiment, the enzyme or mixture of enzymes can be enzyme domains with a particular catalytic activity. For example, an enzyme with multiple activities can have multiple enzyme domains, including for example glycoside hydrolases, glycosyltransferases, lyases and/or esterases catalytic domains.

In one embodiment, enzymes that degrade polysaccharides are used for the hydrolysis of biomass and can include enzymes that degrade cellulose, namely, cellulases. Examples of some cellulases include endocellulases and exo-cellulases that hydrolyze beta-1,4-glucosidic bonds.

In one embodiment, enzymes that degrade polysaccharides are used for the hydrolysis of biomass and can include enzymes that have the ability to degrade hemicellulose, namely, hemicellulases. Hemicellulose can be a major component of plant biomass and can contain a mixture of pentoses and hexoses, for example, D-xylopyranose, L-arabinofuranose, D-mannopyranose, Dglucopyranose, D-galactopyranose, D-glucopyranosyluronic acid and other sugars. In one embodiment, enzymes that degrade polysaccharides are used for the hydrolysis of biomass and can include enzymes that have the ability to degrade pectin, namely, pectinases. In plant cell walls, the cross-linked cellulose network can be embedded in a matrix of pectins that can be covalently cross-linked to xyloglucans and certain structural proteins. Pectin can comprise homogalacturonan (HG) or rhamnogalacturonan (RH).

In one embodiment, hydrolysis of biomass includes enzymes that can hydrolyze starch. Enzymes that hydrolyze starch include alpha-amylase, glucoamylase, beta-amylase, exo-alpha-1,4-glucanase, and pullulanase.

In one embodiment, hydrolysis of biomass comprises hydrolases that can include enzymes that hydrolyze chitin. In another embodiment, hydrolases can include enzymes that hydrolyze lichen, namely, lichenase.

In one embodiment, after pretreatment and/or hydrolysis by any of the above methods the feedstock contains cellulose, hemicellulose, soluble oligomers, monomeric sugars, simple sugars, lignin, volatiles and ash. The parameters of the hydrolysis can be changed to vary the concentration of the components of the pretreated feedstock. For example, in one embodiment a hydrolysis is chosen so that the concentration of soluble C5 saccharides is low and the concentration of lignin is high after hydrolysis. Examples of parameters of the hydrolysis include temperature, pressure, time, concentration, composition and pH.

In one embodiment, the parameters of the pretreatment and hydrolysis are changed to vary the concentration of the components of the pretreated feedstock such that concentration of the components in the pretreated and hydrolyzed feedstock is optimal for fermentation with a microbe such as a yeast or bacterium microbe.

In one embodiment, the parameters of the pretreatment are changed to encourage the release of the components of a genetically modified feedstock such as enzymes stored within a vacuole to increase or complement the enzymes synthesized by biocatalyst to produce optimal release of the fermentable components during hydrolysis and fermentation.

In one embodiment, the parameters of the pretreatment and hydrolysis are changed such that concentration of accessible cellulose in the pretreated feedstock is 10%, 5%, 10%, 12%, 13%, 14%, 15%, 16%, 17%, 19%, 20%, 30%, 40% or 50%. In one embodiment, the parameters of the pretreatment are changed such that concentration of accessible cellulose in the pretreated feedstock is 25% to 35%. In one embodiment, the parameters of the pretreatment are changed such that concentration of accessible cellulose in the pretreated feedstock is 10% to 20%.

In one embodiment, the parameters of the pretreatment are changed such that concentration of hemicellulose in the pretreated feedstock is 10/%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In one embodiment, the parameters of the pretreatment are changed such that concentration of hemicellulose in the pretreated feedstock is 5% to 40%. In one embodiment, the parameters of the pretreatment are changed such that concentration of hemicellulose in the pretreated feedstock is 10% to 30%.

In one embodiment, the parameters of the pretreatment and hydrolysis are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In one embodiment, the parameters of the pretreatment are changed such that concentration of soluble oligomers in the pretreated feedstock is 30% to 90%. In one embodiment, the parameters of the pretreatment and/or hydrolysis are changed such that concentration of soluble oligomers in the pretreated feedstock is 45% to 80%. In one embodiment, the parameters of the pretreatment and/or hydrolysis are changes such that most of the hemicellulose and/or C5 monomers and/or oligomers are removed prior to the enzymatic hydrolysis of the C6/lignin mixture.

In one embodiment, the parameters of the pretreatment and hydrolysis are changed such that concentration of simple sugars in the pretreated feedstock is 1%, 5%, 10%, 12%, 13%, 14%, 15%, 16%, 17%, 19%, 20%, 30%, 40% or 50%. In one embodiment, the parameters of the pretreatment and hydrolysis are changed such that concentration of simple sugars in the pretreated feedstock is 0% to 20%. In one embodiment, the parameters of the pretreatment and hydrolysis are changed such that concentration of simple sugars in the pretreated feedstock is 0% to 5%. Examples of simple sugars include, but are not limited to, C5 and C6 monomers and dimers.

In one embodiment, the parameters of the pretreatment are changed such that concentration of lignin in the pretreated and/or hydrolyzed feedstock is 10/%, 5%, 10%, 12%, 13%, 14%, 15%, 16%, 17%, 19%, 20%, 30%, 40% or 50%.

In one embodiment, the parameters of the pretreatment and/or hydrolysis are changed such that concentration of furfural and low molecular weight lignin in the pretreated and/or hydrolyzed feedstock is less than 10%, 9%, 8%, 7%, 6%, 5%, 4%, 3%, 2%, or 1%. In one embodiment, the parameters of the pretreatment and/or hydrolysis are changed such that concentration of furfural and low molecular weight lignin in the pretreated and/or hydrolyzed feedstock is less than 1% to 2%.

In one embodiment, the parameters of the pretreatment and/or hydrolysis are changed to obtain a low concentration of hemicellulose and a high concentration of lignin. In one embodiment, the parameters of the pretreatment and/or hydrolysis are changed to obtain a high concentration of hemicellulose and a low concentration of lignin such that concentration of the components in the pretreated stock is optimal for fermentation with a microbe such as biocatalyst.

In one embodiment, more than one of these steps can occur at any given time. For example, hydrolysis of the pretreated feedstock and hydrolysis of the oligosaccharides can occur simultaneously, and one or more of these can occur simultaneously to the high conversion of monosaccharides to a fuel or chemical and a higher concentration of lignin residues.

In another embodiment, an enzyme can directly convert the polysaccharide to monosaccharides. In some instances, an enzyme can hydrolyze the polysaccharide to oligosaccharides and the enzyme or another enzyme can hydrolyze the oligosaccharides to monosaccharides.

In another embodiment, the enzymes can be added to the fermentation or they can be produced by microorganisms present in the fermentation. In one embodiment, the microorganism present in the fermentation produces some enzymes. In another embodiment, enzymes are produced separately and added to the fermentation.

In another embodiment, the enzymes of the method are produced by a biocatalyst, including a range of hydrolytic enzymes suitable for the biomass materials used in the fermentation methods. In one embodiment, a biocatalyst is grown under conditions appropriate to induce and/or promote production of the enzymes needed for the saccharification of the polysaccharide present. The production of these enzymes can occur in a separate vessel, such as a seed fermentation vessel or other fermentation vessel, or in the production fermentation vessel where ethanol production occurs. When the enzymes are produced in a separate vessel, they can, for example, be transferred to the production fermentation vessel along with the cells, or as a relatively cell free solution liquid containing the intercellular medium with the enzymes. When the enzymes are produced in a separate vessel, they can also be dried and/or purified prior to adding them to the hydrolysis or the production fermentation vessel. The conditions appropriate for production of the enzymes are frequently managed by growing the cells in a medium that includes the biomass that the cells will be expected to hydrolyze in subsequent fermentation steps. Additional medium components, such as salt supplements, growth factors, and cofactors including, but not limited to phytate, amino acids, and peptides can also assist in the production of the enzymes utilized by the microorganism in the production of the desired products.

Biofuel Plant and Process of Producing Biofuel and Lignin Residues and/or Activated Carbon:

Large Scale Fuel, Chemical, and Activated Carbon Production from Biomass

Generally, there are several basic approaches to producing lignin, fuels and chemical end-products from biomass on a large scale utilizing of microbial cells. In the one method, one first pretreats and hydrolyzes a biomass material that includes high molecular weight carbohydrates to lower molecular weight carbohydrates and a high concentration of lignin residues, and then ferments the lower molecular weight carbohydrates utilizing of microbial cells to produce fuel or other products. In the second method, one treats the biomass material itself using mechanical, chemical and/or enzymatic methods. In all methods, depending on the type of biomass and its physical manifestation, one of the processes can comprise a milling of the carbonaceous material, via wet or dry milling, to reduce the material in size and increase the surface to volume ratio (physical modification). Further reduction in size can occur during hydrolysis depending on the type of mechanisms used to pretreat the feedstock. For example, use of an extruder with one or more screws to physically hydrolyze the biomass will result in a reduction in particle size as well. See, e.g., the process described in U.S. provisional patent application No. 62/089,704.

In one embodiment, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrolysis and/or steam treatment of the biomass can, e.g., increase porosity and/or surface area of the biomass, often leaving the cellulosic and lignaceous materials more exposed to the enzymes, which can increase hydrolysis rate and yield of sugars and lignin. Removal of lignin following hydrolysis can result in a low sulfur, low ash, and high porosity lignin residue for the production of activated carbon and other products. The lignin residues can comprise 50% or more of solid particles. Depending on feedstock composition, the lignin residues will contain at least 50% of solid particles from about 5 microns to about 150 microns in size. More typically, but depending on feedstock composition, lignin residues of a pretreated biomass wherein the lignin residues comprise at least 50% of solid particles from about 5 microns to about 150 microns in size.

In one embodiment, the activated carbon produced from lignin residues will have relatively high carbon content/unit mass as compared to the initial feedstock because much of the non-lignin material, including the carbon bonded to the hemicellulose, cellulose, proteins, oils and salts will be removed through the hydrolysis and separation processes. An activated carbon as provided herein will normally contain greater than about half its weight as carbon, since the typical carbon content of biomass is no greater than about 50 wt % and the remaining lignin residues will be reduced in many elements. More typically, but depending on feedstock composition, an activated carbon will contain at least 55 wt %, at least 60 wt %, at least 65 wt %, at least 70 wt %, at least 75 wt %, at least 80 wt % 85 wt %, at least 90 wt %, at least 95 wt %, at least 96 wt %, at least 97 wt %, at least 98 wt %, at least 99 wt % carbon.

Biomass Processing Plant and Process of Producing Products from Biomass

In one aspect, a fuel or chemical plant or system that includes a pretreatment unit to prepare biomass for improved exposure and biopolymer separation, a hydrolysis unit configured to hydrolyze a biomass material that includes a high molecular weight carbohydrate, and one or more product recovery system(s) to isolate a product or products and associated by-products and lignin co-products is provided. In another aspect, the pretreatment unit produces a pretreated biomass composition comprising solid particles, C5 and C6 polymers, monomers and dimers by hydrating the biomass composition in a non-neutral pH aqueous medium to produce a hydrated biomass composition that is reduced in size heating the biomass composition under pressure for a time sufficient to produce carbohydrate monomers and oligomers and lignin residues. In another aspect, methods of purifying lower molecular weight carbohydrate from solid byproducts and/or toxic impurities are provided.

In one aspect the biomass processing plant or system includes an enzymatic hydrolysis unit to produce a sugar stream and a residual solids that contain lignin residues. The enzymatic hydrolysis is preceded by neutralizing the pretreated hydrolysis product by adjusting the pH to a range of pH 4.5 to pH 6.5, preferably about pH 5.5 for optimal cellulolytic and hemicellulolytic hydrolysis. The pH-adjusted hydrolysis product is then enzymatically hydrolyzed by isolated enzymes or other biocatalysts for a period of time to hydrolyze the carbohydrate polymers to monomers. In one embodiment, a biocatalyst includes microorganisms that hydrolyze carbohydrate polymers to oligomers and monomers. Lignin residues are further separated from bound carbohydrate through this process.

In another aspect, methods of making a product or products that include combining biocatalyst cells of a microorganism and a biomass feed in a medium wherein the biomass feed contains lower molecular weight carbohydrates and unseparated solids and/or other liquids from pretreatment and hydrolysis, and fermenting the biomass material under conditions and for a time sufficient to produce a biofuel, chemical product or fermentive end-products, e.g. ethanol, propanol, hydrogen, succinic acid, lignin, terpenoids, and the like as described above, is provided. The pretreated biomass is contacted with the enzyme mix or microorganisms, or both for sufficient time to product a sugar stream and lignin residues.

In another aspect, a separation unit is provided that comprises a means to separate the lignin residues from the sugars, proteins, any products formed, and other materials. Separation can occur by means of filtration, flocculation, centrifugation, and the like.

In another aspect, a carbon chemical plant that includes a carbonization unit to prepare high-porous carbon from lignin co-products and residues, and further provides an activation unit to activate the carbon produced from the lignin co-products and residues is provided. In another aspect, the carbon chemical plant is made a part of the fuel or chemical plant so that lignin co-products and lignin residues are easily transported to the carbon chemical plant. In another aspect, the carbon chemical plant is provided with a shaping unit to process the activated carbon into powdered activated carbon (PAC), granular activated carbon (GAC), extruder activated carbon (EAC), graphite, pellets or cylinders, or a combination thereof, or another form. In another aspect, the carbon is further processing to produce an impregnated activated carbon.

In another aspect, products made by any of the processes described herein are also provided herein.

This system can be constructed so that all of the units are physically close, if not attached to one and other to reduce the costs of transportation of a product. For example, the pretreatment, enzymatic hydrolysis, separation, carbonization and activation unit can all be located at a sawmill or agricultural site. Not only is the cost of transporting the biomass to the pretreatment unit virtually eliminated, the lignin residues are processed in the carbonization and activation units, thus eradicating the cost of shipping the lignin residues. Thus, in addition to sugars, sugar products, fuels, such as ethanol, and other biochemcals, the same processing facility can produce activated carbon for many different uses.

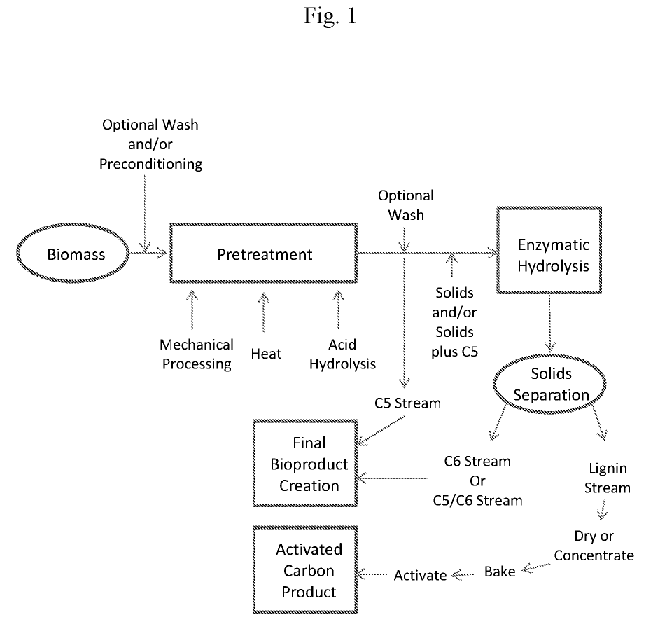

FIG. 1 is an example of a method for producing sugar streams and lignin residues from biomass by first treating biomass with an acid at elevated temperature and pressure in a thermal/chemical hydrolysis unit. The biomass may first be heated by addition of hot water or steam. The biomass may be acidified by bubbling gaseous sulfur dioxide through the biomass that is suspended in water, or by adding a strong acid, e.g., sulfuric, hydrochloric, or nitric acid with or without preheating/presteaming/water addition. Weaker acids or organic acids, such as carbonic, oxalic, malic, and the like can also be used. During the acidification, the pH is maintained at a low level, e.g., below about 5. The temperature and pressure may be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the acid hydrolysis of the biomass. The acid-impregnated biomass can be fed into the hydrolysis section of the pretreatment unit. Steam is injected into the hydrolysis portion of the pretreatment unit to directly contact and heat the biomass to the desired temperature and/or pressure. The temperature of the biomass after steam addition can be, e.g., from about 130° C. to 220° C. The acid hydrolysate can then be discharged into the flash tank portion of the pretreatment unit, and can be held in the tank for a period of time to further hydrolyze the biomass, e.g., into oligosaccharides and monomeric sugars. Other methods can also be used to further break down biomass. Hydrolysate can then be discharged from the pretreatment reactor, with or without the addition of water, e.g., at solids concentrations from about 10% to about 60%.

After physical hydrolysis pretreatment, the biomass may be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products may be derived from treatment of the acidified fluid, e.g., gypsum or ammonium phosphate.

Wash fluids can be collected to concentrate the C5 saccharides in the wash stream. At such a point, the solids can be separated from the C5 stream and the C5 stream further purified.

Enzymes or a mixture of enzymes can be added during pretreatment to hydrolyze, e.g., endoglucanases, exoglucanases, cellobiohydrolases (CBH), beta-glucosidases, glycoside hydrolases, glycosyltransferases, alphyamylases, chitinases, pectinases, lyases, and esterases active against components of cellulose, hemicelluloses, pectin, and starch, in the hydrolysis of high molecular weight components. If the C5 saccharides are not collected separately, they are included in the enzymatic hydrolysis of the stream. Thus enzymatic hydrolysis can produce a fairly pure C6 stream or a mixed C5 and C6 stream. Solids can then be removed, and the C6 or the mixed stream can then be further refined. If the sugar stream is not concentrated, it can be further concentrated, for example, through evaporation.

In some embodiments, the isolated sugar stream has a pH of from about 4 to about 5.5, from about 4.5 to about 5, about 4, about 4.5, about 5, about 6, about 5.5 or more.

In some embodiments, the carbohydrate is contained in the sugar stream in an amount of: about 1% w/v to about 60% w/v, about 1% w/v to about 50% w/v, about 1% w/v to about 40% w/v, about 1% w/v to about 30% w/v, about 1% w/v to about 20% w/v, about 1% w/v to about 10% w/v, about 2% w/v, about 3% w/v, about 4% w/v, about 5% w/v, about 6% w/v, about 7% w/v, about 8% w/v, about 9% w/v, about 15% w/v, about 25% w/v, about 35% w/v, or about 40% w/v.

In some embodiments, the isolated sugar stream comprises C5 sugars, C6 sugars, or a combination thereof.

In some embodiments, the amount of sugar in the sugar stream is: about 1% w/v to about 60% w/v, about 1% w/v to about 50% w/v, about 1% w/v to about 40% w/v, about 1% w/v to about 30% w/v, about 1% w/v to about 20% w/v, about 1% w/v to about 10% w/v, about 5% w/v, about 15% w/v, about 25% w/v, about 35% w/v, about 45% w/v, or about 55% w/v.

In some embodiments is provided a method of producing a sugar stream comprising C5 and C6 sugars from a biomass composition comprising cellulose, hemicellulose, and/or lignocellulose, the method comprising:

(a) pretreating the biomass composition comprising cellulose, hemicellulose, and/or lignocellulose to produce a pretreated biomass composition comprising solid particles and optionally a yield of C5 monomers and/or dimers that is at least 50% of a theoretical maximum, wherein pretreating comprises:

(i) hydration of the biomass composition in a non-neutral pH aqueous medium to produce a hydrated biomass composition,

(ii) mechanical size reduction of the hydrated biomass composition to produce the solid particles, and

(iii) heating the hydrated biomass composition for a time sufficient to produce the pretreated biomass composition comprising the optional yield of C5 monomers and/or dimers or oligomers that is at least 50% of the theoretical maximum;

(b) hydrolyzing the pretreated biomass composition with one or more enzymes for a time sufficient to produce the composition comprising C6 and C5 sugars;

(c) washing the hydrolyzed biomass results in recovery a sugar stream substantially enriched for C6 and/or C5 sugars; and

In some embodiments, at least 50% of the solid particles in the pretreated biomass composition are from about 3.0 microns to about 150 microns in size.

In some embodiments, all of the solid particles in the pretreated biomass are less than 1.0 mm in size.

In some embodiments, all of the solid particles in the pretreated biomass are less than 0.1 mm in size.

In some embodiments, the pretreated biomass composition further comprises a yield of glucose that is less than about 25% of the theoretical maximum.

In some embodiments, the hydrated biomass composition comprises from about 10% to about >40% solids by dry biomass weight.

In some embodiments, the non-neutral pH aqueous medium is at from about 70° C. to above 100° C.

In some embodiments, hydration of the biomass composition is for about 1 minute to about 60 minutes prior to hydrolysis.

In some embodiments, the non-neutral aqueous medium comprises an acid or a base at from about 0.1% to about 5% v/w by dry biomass weight.

In some embodiments, the non-neutral pH aqueous medium comprises the acid that is sulfuric acid, peroxyacetic acid, lactic acid, formic acid, acetic acid, citric acid, phosphoric acid, hydrochloric acid, sulfurous acid, chloroacetic acid, dichloroacetic acid, trichloroacetic acid, trifluoroacetic acid, oxalic acid, benzoic acid, or a combination thereof.

In some embodiments, mechanical size reduction comprises cutting, grinding, steam injection, steam explosion, acid-catalyzed steam explosion, ammonia fiber/freeze explosion (AFEX) or a combination thereof.

In some embodiments, heating of the hydrated biomass composition is at a temperature of from about 100° C. to about 250° C.

In some embodiments, heating of the hydrated biomass composition is performed at a pressure of from about 100 PSIG to about 750 PSIG, more particularly 400 PSIG to 500 PSIG.

In some embodiments, the time sufficient to produce the yield of C5 monomers and/or dimers is from about 10 sec to about 30 sec.

In some embodiments, pretreating the biomass composition further comprises dewatering the hydrated biomass composition to from about 10% to about 40% solids by dry biomass weight.

In some embodiments, heating comprises steam explosion, acid-catalyzed steam explosion, ammonia fiber/freeze explosion (AFEX), or a combination thereof.

In some embodiments, the pretreating is performed in a continuous mode of operation.

In some embodiments, the method further comprises adjusting the water content of the pretreated biomass composition to from about 5% to about 30% solids by dry biomass weight prior to hydrolyzing.

In some embodiments, the biomass composition comprises alfalfa, algae, bagasse, bamboo, sorghum, corn stover, corncobs, corn fiber, corn kernels, corn mash, corn steep liquor, corn steep solids, distiller's grains, distiller's dried solubles, distiller's dried grains, condensed distiller's solubles, distiller's wet grains, distiller's dried grains with solubles, eucalyptus, food waste, fruit peels, garden residue, grass, grain hulls, modified crop plants, municipal waste, oat hulls, paper, paper pulp, prairie bluestem, poplar, rice hulls, seed hulls, almond shells, peanut shells, coconut shells, silage, sorghum, straw, sugarcane, switchgrass, wheat, wheat straw, wheat bran, de-starched wheat bran, willows, wood, sawdust, wood chips, plant cells, plant tissue cultures, tissue cultures, or a combination thereof.

In some embodiments, the sugar stream comprises water, an alcohol, an acid, or a combination thereof and the lignin residues comprise lignin, sugar monomers, saccharide oligomers, minerals, protein and enzymes.

In some embodiments, the sugar stream is subjected to an enzymatic hydrolysis prior to separation of the lignin residues.

In some embodiments, the sugar stream and lignin residues are derived from a biomass.

In some embodiments, the biomass is pretreated.

In some embodiments are provided an isolated sugar stream and lignin residues produced by the method of any one of the above embodiments.

In some embodiments is provided a system for producing a sugar stream consisting of C5 and C6 saccharides and lignin residues by the method of any previous method embodiment.

In some embodiments, the separation of lignin residues from the sugar stream is by means of a flocculation, a filtration, a centrifugation, or any combination thereof.

Production of Activated Carbon

The lignin residues can also be concentrated by any means, such as drying, evaporation, flocculation, filtration, centrifugation or a combination of these methods. They are usually dried and can be shaped into pellets, bricks, or any desirable shape. In one embodiment, the lignin residues can be crumbled or ground into a powder.

In one embodiment, a unit is provided for carbonization and activation to convert the lignin residues into activated carbon.

In another embodiment, the concentrated lignin residues are shipped to a different site for conversion to activated carbon.

Carbonization and Activation:

In any shape, or in powdered or granulated form, lignin residues are carbonized to produce a char in a furnace, such as a rotary furnace, via fluidized bed, rotary kiln, extruder, or any other means of heating to an adequate temperature. Residues are heated to at least about 200° C. and above 300° C. to about 700° C. Preferably, the residues are heated to at least 200° C. and less than 350° C.

Toward the end of the carbonizing cycle, or following this cycle, the lignin residues are also preferably activated in the furnace by heating to 800° C. or higher and preferably 800° C. to 1800° C. Chemical activation can be completed at lower temperatures ranging from about 300° C. to 900° C.

Once the porous form of carbon is produced, it typically undergoes oxidization so it can be adsorbent. This can occur. e.g., m one of two ways: physical or chemical activation.

Physical activation of carbon can be done directly through heating in a chamber while gas is pumped in, typically CO₂ or steam. This exposes it to oxygen for oxidization purposes. When oxidized, the active carbon can be susceptible to adsorption, the process of surface bonding for chemicals which is the very thing that makes activated carbon so good for filtering waste and toxic chemicals out of liquids and gases. For physical gas treatment, the carbonization pyrolysis process can take place in an inert environment at 200-900° C. Later, an oxygenated gas can be pumped into the environment and heated between 700° C. and 1200° C. or higher, causing the oxygen to bond to the carbon's surface.

In chemical activation, the process is slightly different from the physical activation of carbon. For one, carbonization and chemical activation occur simultaneously. In one embodiment, a bath of acid, base or other chemicals is prepared and the material submerged. The material soaks up the chemical and is then “chemically charged” to activate the carbon and further dried by heating to temperatures of 400°-900° Celsius, much less than the heat needed for physical activation. Chemicals useful for chemical activation include, but are not limited to, ZnCl₂, H₃PO₄, Na₂CO₃, K₂CO₃, and some alkali metal compounds. In this process, the carbonaceous material is carbonized and then activated all at a much quicker pace than physical activation. However, some heating processes cause trace elements from the bath to adsorb to the carbon, which can result in impure or ineffective active carbon in the presence of material selected from the group consisting of steam, acid, carbon dioxide and/or flue gas and the like. In an alternate embodiment, chlorine or similar gases or vapors may be utilized at high temperature or air at low temperature to selectively oxidize and activate the separated agglomerates. On completion of the carbonizing and activating cycles, the activated carbon is removed from furnace, kiln, fluidized bed or other means of carbonization and/or activation as a finished product.

Post Treatment

Following oxidization, activated carbon can be processed for many different kinds of uses, with several classifiably different properties. For instance, granular activated carbon (GAC) is a sand-like product with bigger grains than powdered activated carbon (PAC), and each can be used for different applications. Other varieties include impregnated carbon, which includes different elements such as silver and iodine, and polymer-coated carbons. Applications of impregnated activated carbon include bottled water and beverage production, drinking water treatment, groundwater remediation, industrial process water, odor and vapor control and wastewater treatment.

Preferably the PAC has a particle size of: from about 5 microns to about 40 microns, about 5 microns to about 30 microns, about 5 microns to about 20 microns, less than about 40 microns, less than about 30 microns, less than about 20 microns, less than about 10 microns, or less than about 5 microns.

In some embodiments, the activated carbon has a particle size of: from about 5 microns to about 40 microns, about 5 microns to about 30 microns, about 5 microns to about 20 microns, less than about 40 microns, less than about 30 microns, less than about 20 microns, less than about 10 microns, or less than about 5 microns.

In some embodiments, the activated carbon has a particle size ranging from about 5 microns to about 0.177 mm.

In some embodiments, the carbonization is conducted at a temperature of: about 200° C. to about 300° C., about 250° C. to about 350° C., about 350° C. to about 600° C., about 600° C. to about 800° C., or about 850° C. to about 900° C.

In some embodiments, the carbonization is conducted for a time period of: 30 sec to about 1 min, about 1 min to about 5 min, about 5 min to about 1 hour, about 1 hour to about 24 hours, about 1 hour to about 18 hours, about 1 hour to about 12 hours, about 1 hour to about 6 hours, about 2 hours, about 3 hours, about 4 hours, about 6 hours, about 7 hours, about 8 hours, about 9 hours, about 10 hours, about 11 hours, about 13 hours, about 14 hours, about 15 hours, about 17 hours, about 19 hours, about 20 hours, about 21 hours, about 22 hours, or about 23 hours.

In some embodiments, the carbonization and activation are done simultaneously.

In some embodiments, the heating of carbonization is conducted at a temperature of: about 150° C. to about 300° C., about 150° C. to about 250° C., about 150° C. to about 200° C., about 160° C., about 170° C., about 180° C., about 190° C., about 210° C., about 220° C., about 230° C., about 240° C., about 260° C., about 270° C., about 280° C., or about 290° C., about 300° C., about 350° C., about 400° C., about 450° C., about 500° C., about 550° C., about 600° C., about 650° C., about 700° C., about 750° C., about 800° C., about 850° C., about 900° C., about 950° C., about 1000° C., about 1100° C., about 1200° C., about 1300° C., about 1400° C., about 1500° C., about 1600° C., about 1700° C., or about 1800.

In some embodiments, the heating of carbonization is conducted under vacuum.

In some embodiments, the activated carbon is powdered activated carbon (PAC), granular activated carbon (GAC), extruded activated carbon (EAC), and bead activated carbon (BAC), graphite, impregnated activated carbon, or a combination thereof.

In some embodiments, the activated carbon has a particle size of: from about 5 microns to about 40 microns, about 5 microns to about 30 microns, about 5 microns to about 20 microns, less than about 40 microns, less than about 30 microns, less than about 20 microns, less than about 10 microns, or less than about 5 microns.

In some embodiments, the activated carbon, before the contacting, is activated by heating.

In some embodiments, the heating is conducted for a time period of: about 30 sec to about 10 min, about 1 min to about 20 min, about 20 min to about 1 hour, about 1 hour to about 48 hours, about 1 hour to about 36 hours, about 1 hour to about 24 hours, about 1 hour to about 18 hours, about 1 hour to about 12 hours, about 1 hour to about 6 hours, about 2 hours, about 3 hours, about 4 hours, about 6 hours, about 7 hours, about 8 hours, about 9 hours, about 10 hours, about 11 hours, about 13 hours, about 14 hours, about 15 hours, about 17 hours, about 19 hours, about 20 hours, about 21 hours, about 22 hours, or about 23 hours.

In some embodiments, the activation is conducted at a temperature of: about 150° C. to about 300° C., about 150° C. to about 250° C., about 150° C. to about 200° C., about 160° C., about 170° C., about 180° C., about 190° C., about 210° C., about 220° C., about 230° C., about 240° C., about 260° C., about 270° C., about 280° C., or about 290° C., or about 300° C., or about 350° C., or about 400° C., or about 450° C., or about 500° C., or about 550° C., or about 600° C., or about 650° C.

In some embodiments, the heating is conducted under vacuum.

In some embodiments, is provided a system comprising a pretreatment unit, configured to pretreat a biomass by at least one of mechanical processing, heat, acid hydrolysis, steam explosion or any combination thereof, and an enzymatic hydrolysis unit configured to hydrolyze saccharide polymers to saccharide monomers and oligomers and then to a product, a separation unit configured to separate a product of enzymatic hydrolysis from lignin residues, a carbonization unit configured to convert lignin to carbon (char), and an activated carbon unit configured to convert carbon (char) into activated carbon.

In some embodiments, the system further comprises, upstream of the pretreatment unit, a preconditioning unit configured to clean, condition and hydrate a biomass before the biomass is fed to the pretreatment unit.

In some embodiments, the system further comprises, upstream of the hydrolysis unit and downstream of the pretreatment unit, a washing unit configured to wash pretreated biomass before the pretreated biomass is fed to the hydrolysis unit.

In another aspect, the products made by any of the processes described herein is provided.

### Examples

The following examples serve to illustrate certain embodiments and aspects and are not to be construed as limiting the scope thereof.

### Example 1. Pretreatment of Biomass

A twin screw extruder was used to perform four continuous runs of 224, 695, 1100, and 977 hours each on corn fiber. The extruder was run with indirect heating through the reactor walls until the end of the experiment. A flow rate of up to 300 lb/hr (136 kg/hr) was reached through the extruder, with direct steam injection to supply process heat. The materials selected were acid resistant. The feed was metered through a weight belt feeder and fell into a crammer feeder supplying the barrel of the extruder. Two screws intermeshed and provided rapid heat and mass transfer when steam and sulfuric acid were injected through steam and acid ports connected to the cylindrical barrel of the extruder. The steam and acid supplying ports were sealed by reverse-flow sections in the screws. A hydraulically operated pressure control valve was seated in a ceramic seal and pressure was controlled to maintain as constant a pressure as possible in the reaction section of the extruder.

The solids were exposed to high temperature and pressure and low pH for a maximum of about 10 seconds in the reaction zone of the extruder before being exploded into the flash tank. Residence time in the reaction zone was controlled by the feed rate and the rotational speed of the screws. The surge chamber above the screws in the pump feeder acted as a flash vessel, where hot water is vaporized, cooling the product and removing some of the low-boiling inhibitors, such as furfural. HMF and furfural, reversion inhibitors, were formed in small amounts during this pretreatment (e.g., a total of 0.3 to 0.5 wt. % of the dry pretreated product).

A mixture of different enzymes were used to hydrolyze the remaining cellulose and hemicellulose into C5 and C6 saccharides in the hydrolysis product following the addition of water and neutralization of the mixture to about pH 5.0. Following enzymatic hydrolysis for 48-56 hrs, the remaining solids, including lignin, were flocculated and separated from the solubilized sugars by filtration. The remaining lignin residues were dried.

### Example 2. Lignin Sample EE-643 Conversion to Activated Carbon

A sample of lignin residue was charred by heating at 150° C. for three hours during which time it lost 37.1% weight. An additional three hours at 150° C. resulted in an additional 4.9% weight loss. The dry material was crushed and screened with standard sieves. The dry apparent density of the sample was 0.3621 g/cc and the Dean-stark moisture % was 43.1. Crushed lignin sample (108 ml) was screened with standard sieves to give the following analysis:

| 6 | 8 | 12 | 16 | 20 | 30 | <30 | U.S. sieve number |
|---|---|---|---|---|---|---|---|
| 9.11 | 39.6 | 37.23 | 10.26 | 1.93 | 1.06 | 0.81 | Grams on sieve |

| | | As Received | Dry | Dry Ash-Free |
|---|---|---|---|---|
| Proximate Analysis | | | | |
| Moisture | D3172 | 52.44 | | |
| Ash | | 0.43 | 0.90 | |
| Volatile Matter | | 32.66 | 68.67 | 69.30 |
| Fixed Carbon | | 14.47 | 30.43 | 30.70 |
| | | 100.00 | 100.00 | 100.00 |
| Ultimate Analysis | | | | |
| Hydrogen | D5373 | 8.57 | 5.67 | 5.72 |
| Carbon | D5373 | 28.12 | 59.12 | 59.65 |
| Nitrogen | D5373 | 0.32 | 0.67 | 0.68 |
| Sulfur | D4239-02 | 0.10 | 0.22 | 0.22 |
| Oxygen | D3176 | 62.46 | 33.42 | 33.73 |
| Ash | D3174-02 | 0.43 | 0.90 | |
| | | 100.00 | 100.00 | 100.00 |

| | | Apparent | | Carbon Tetra |
|---|---|---|---|---|
| | Yield | Density | Iodine | Chloride |
| Activation | Family % | g/cc | No., mg/g | CTC g/100 g C |
| 5 minutes | 26 | 0.31 | 300 | 16.21 |
| 11 | 27 | 0.29 | 715 | 35.21 |
| 16 | 24 | 0.27 | 943 | 50.22 |
| 22 | 22 | 0.26 | 1064 | 59.78 |

Proximate and Ultimate analysis and activation data is as follows:

These samples show a very low ash and sulfur content and a high oxygen content.

### Example 3. Lignin Sample EE-634

Lignin material was activated in lab sized rotary kiln to make enough material for test methods. Baking was provided in a muffle furnace to more closely mimic commercial production. Material with 1,000- and 500-Iodine was produced; there were seven kiln runs per Iodine target. Duration of activation was 22 minutes for 1,000 and 6 minutes for 500-Iodine.

Results are below:

| Run | Activation 22 minutes | | | Activation 6 minutes | | |
|---|---|---|---|---|---|---|
| Number | Iodine | Density | Yield % | Iodine | Density | Yield % |
| 1 | 1070 | 0.27 | 46 | 500 | 0.31 | 50 |
| 2 | 1090 | 0.26 | 45 | 520 | 0.32 | 51 |
| 3 | 1045 | 0.25 | 42 | 470 | 0.30 | 49 |
| 4 | 1020 | 0.27 | 47 | 525 | 0.33 | 52 |
| 5 | 1095 | 0.26 | 44 | 505 | 0.32 | 47 |
| 6 | 1005 | 0.27 | 43 | 525 | 0.31 | 51 |
| 7 | 1020 | 0.27 | 45 | 465 | 0.33 | 52 |

Screen Sizing Data - Sieve Analysis 1,000 Iodine Sample EE-634

| 6 | 8 | 12 | 16 | 20 | 30 | <30 | U.S. sieve number |
|---|---|---|---|---|---|---|---|
| 3.1 | 43.9 | 39.8 | 6.21 | 3.75 | 2.24 | 1.1 | Grams on sieve |

Screen Sizing Data - Sieve Analysis 500 Iodine Sample EE-634

| 6 | 8 | 12 | 16 | 20 | 30 | <30 | U.S. sieve number |
|---|---|---|---|---|---|---|---|
| 4.91 | 47.8 | 40.7 | 40.7 | 1.0 | 0.03 | 0.66 | Grams on sieve |

| ASTM | ASTM | |
|---|---|---|
| Contact pH Rise | Hardness 1000 Iodine | Hardness 500 Iodine |
| 0.92 | 73 | 78 |

| Benchmark Vapor Phase | SWE 500 Iodine |
|---|---|
| Mercury Capacity | Mercury Capacity |
| 1514 | 1450 |

| 1,000 Iodine EE-634 | | | | |
|---|---|---|---|---|
| Proximate Analysis | D3172 | As Received | Dry | Dry Ash-Free |
| Moisture | | 7.91 | | |
| Ash | | 2.48 | 2.69 | |
| Volatile Matter | | 3.22 | 3.50 | 3.59 |
| Fixed Carbon | | 86.39 | 93.81 | 96.41 |
| | | 100.00 | 100.00 | 100.00 |

| 1,000 Iodine EE-634 | | | | |
|---|---|---|---|---|
| Ultimate Analysis | | | | |
| Hydrogen | D5373 | 0.74 | 0.00 | 0.00 |
| Carbon | D5373 | 85.16 | 92.48 | 95.04 |
| Nitrogen | D5373 | 0.58 | 0.63 | 0.64 |
| Sulfur | D4239-02 | 0.14 | 0.16 | 0.16 |
| Oxygen | D3176 | 10.90 | 4.04 | 4.16 |
| Ash | D3174-02 | 2.48 | 2.69 | |
| | | 100.00 | 100.00 | 100.00 |

| 500 Iodine EE-634 | | | | |
|---|---|---|---|---|
| Proximate Analysis | D3172 | As Received | Dry | Dry Ash-Free |
| Moisture | | 5.78 | | |
| Ash | | 2.61 | 2.77 | |
| Volatile Matter | | 3.09 | 3.28 | 3.37 |
| Fixed Carbon | | 88.52 | 93.95 | 96.63 |
| | | 100.00 | 100.00 | 100.00 |

| 500 Iodine | | | | |
|---|---|---|---|---|
| Ultimate Analysis | EE-634 | As Received | Dry | Dry Ash-Free |
| Hydrogen | D5373 | 0.51 | 0.00 | 0.00 |
| Carbon | D5373 | 86.91 | 92.24 | 94.87 |
| Nitrogen | D5373 | 0.73 | 0.77 | 0.80 |
| Sulfur | D4239-02 | 0.17 | 0.19 | 0.19 |
| Oxygen | D3176 | 9.07 | 4.03 | 4.14 |
| Ash | D3174-02 | 2.61 | 2.77 | |
| | | 100.00 | 100.00 | 100.00 |

A longer activation period would produce a higher Iodine number. The 22 minute activation made the best Iodine product at 1064. The potential is 1,300 to 1,500 Iodine.

Calcium bromide can be added to this activated carbon to increase commercial product's ability to capture vapor phase mercury. Commercial products can add about 5% weight/weight of Calcium bromide. The 500 Iodine product is about 95% of the benchmark for a commercial product for vapor phase mercury capacity, which can be enhanced with Calcium bromide.

### Example 4. Lignin Sample EE-634A2 Vapor Phase Comparisons

A carbonaceous sample of lignin residues was prepared for activation by stage grinding the waffle-like material, baking it and then steam activating a progressive series at 850° C. based on different times. One sample of this granular activated carbon (GAC) was chosen for full characterization for aqueous phase comparison using the Gravimetric Adsorption Energy Distribution method (GAED). The sample lignin (EE-634A2), was activated for 22 minutes to an Apparent Density (AD) of 0.265 g/cc and had the highest activity (Iodine # of 1064 mg/g) of the four activations. It was then compared to four commercially available carbons: BPL Coal-based gas phase, BG-HHM Wood-based, CAL Coal-based liquid phase and PCB Coconut-based carbon of about 1200 iodine number. The AD was determined by using the ASTM D-2854-96 and made volume-based comparisons possible. The sample lost over 7 weight percent on conditioning (heating the sample to 240° C. in argon and holding for 25 minutes) indicating it had picked up some water weight upon discharge from the kiln. The conditioned sample showed a little over 93% of the total adsorption pore volume as that of CAL, Coal-based Liquid phase reference material. The calculated BET surface was 703 sq.meters/g, which is about 80% of the PCB Reference material. The structural of this sample, as seen in the Differential Characteristic Curves, was more like that of the BG-HHM wood-based reference and had an increased pore structure at the larger pore areas. This sample showed its best potential in good trace capacity activity compared to the other reference samples for calculated Isotherms of MTBE, Benzene and Phenol. In the six Application Performance graphs, its best performance would be in specific applications of Type IV (Regenerable Trace Loading Applications like Acetone Solvent Recovery), Type V (Trace Loading Applications like Trichloroethane from Water) and Type VI (Ultra Trace Loading Applications like Vinyl Chloride from Water). The activation study used lab scale equipment, had about 20% overall yield but is not an optimization trial.

GAED Results:

The waffle-like lignin material was stage ground and sized to 3×12 mesh, baked and then activated at 850° C. at four different times creating EE-634A1, EE-634A2, EE-634A3 and EE-634A4. One sample was chosen for full characterized of aqueous phase comparison by the GAED (gravimetric adsorption energy distribution method).

Sample EE-634A2 was fully characterized for aqueous-phase GAED by measuring the entire characteristic curves using the GAED. The Apparent Density (AD) of 0.265 g/cc was used allowing volume-based results. The carbons were then compared to four commercially activated reference samples made from a range of raw materials.

The sample was in a raw carbon form when received. In preparation, this material was sized, baked then activated at 850° C. A summary of the actual GAED test data and conditions used is listed in the data summary Table 1.

The lignin EE-634A2 sample lost 7.44 weight percent on conditioning (heating to 240° C. in argon and holding for 25 minutes). Losses of less than 8 percent indicate a well-stored sample that has been protected from the small amount of moisture pick-up from ambient air during handling and storage. The sample weight loss was undoubtedly due to water pickup at discharge from the kiln. This sample had no chance to be exposed to contaminants and was protected, was fresh and not oxidized. All activities and adsorption capacities were calculated on a clean carbon basis.

Sample identification is as follows: EE624A2: BPL coal-base gas phase; BG-HHM wood base; CAL coal-base liquid phase; PCB coconut-base.

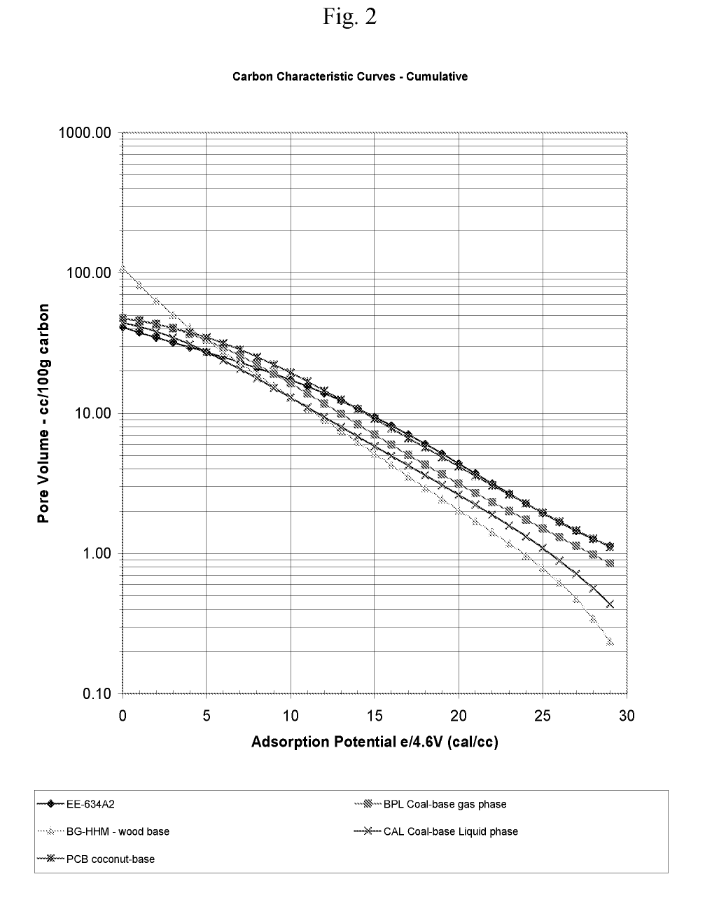

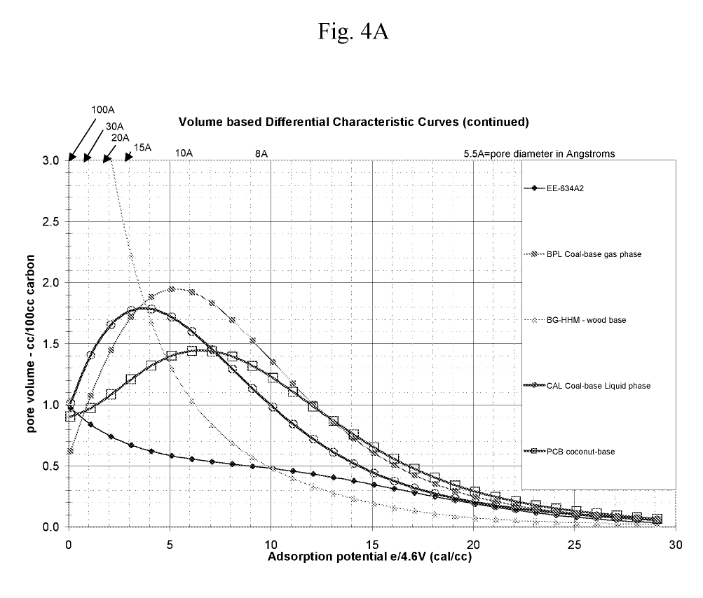

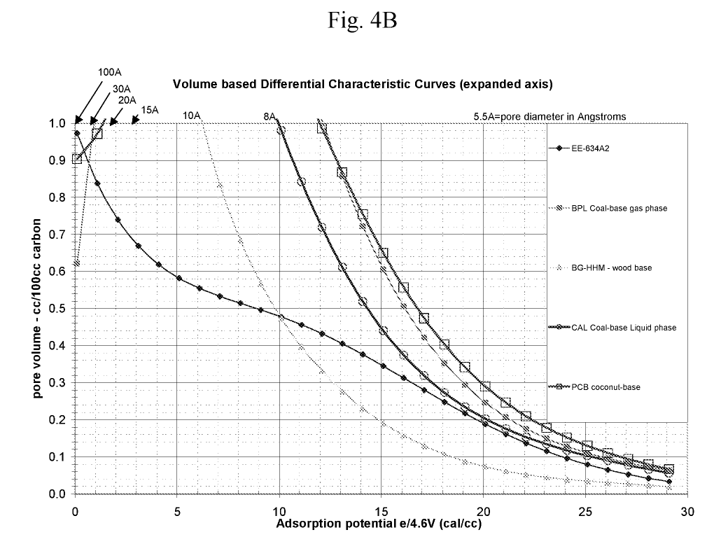

The GAED run was typical. The difference between the adsorption and desorption curves was minor throughout the experiment, therefore no hysteresis was present, as was normal for commercially activated carbons. The plots of the differential and cumulative characteristic curve data are presented in FIGS. 2, 4A and 4B in a volume-based comparison.

TABLE 1
EE-634A2

| Sample Description | | | Carbon Characteristic Curve | | |
|---|---|---|---|---|---|
| EE-634A2 | | | EE-634A2 | | |
| My Act (EE-634A2) | | | Adsorption | | |
| 0.265 g/cc AD | | | Potential | Differential | Cumulative |
| Equipment Information | | Calculated N2 BET Surface Area | e/4.6 V | Pore Volume | Pore Volume |
| Operator CDM | | BET sq. meters/g = 703 | (cal/cc) | cc/100 g | cc/100 g |
| Analysis Date May 13, 2015 | | BET C Constant = −92.1824 | 0 | 3.67 | 40.96 |
| Start time 1:55:12 PM | | Max. P/Po = 0.298 | 0.4 | 3.45 | 39.53 |
| Procedure Auto GAED ver. 10/09 | | Min. P/Po = 0.051 | 1 | 3.16 | 37.54 |
| File C:\data\PACS | | R square = 0.9961 | 1.4 | 3.00 | 36.30 |
| OrgFile C:\data\PACS | | Single point BET sq. meters/g = 699 | 2 | 2.79 | 34.55 |
| Instrument GAED | | | 3 | 2.52 | 31.89 |
| Module Mettler | | | 4 | 2.33 | 29.46 |
| Xcomment Pan:Al - Gas1:Argon - Gas2:C134a 100 cc/min | | | 5 | 2.19 | 27.19 |
| Text 500 mg Al pan full level - Straight TC | | | 6 | 2.09 | 25.05 |
| | | | 7 | 2.01 | 22.99 |
| | | | 8 | 1.94 | 21.01 |
| Conditioning the Sample | | | 9 | 1.87 | 19.11 |
| Pan:Al - Gas1:Argon Conditioning gas | | | 10 | 1.80 | 17.27 |
| 236.3 C. Conditioning temperature in Argon | | | 11 | 1.72 | 15.50 |
| 0.9025 g Original Carbon wt | | | 12 | 1.63 | 13.82 |
| 0.8418 g Clean carbon weight | | | 13 | 1.53 | 12.24 |
| 7.44% wt % loading unconditioned | | | 14 | 1.42 | 10.76 |
| | | | 15 | 1.30 | 9.40 |
| Adsorption/desorption experiments | | | 16 | 1.18 | 8.15 |
| 5 Deg/min adsorption/desorption | | | 17 | 1.06 | 7.03 |
| Gas2:C134a 100 cc/min Adsorbate gas | | | 18 | 0.93 | 6.02 |
| −8.56 C. Minimum adsorption temperature | | | 19 | 0.82 | 5.14 |
| 438 Number of data points | | | 20 | 0.71 | 4.38 |
| 3 pnts/min Data collection rate | | | 21 | 0.61 | 3.71 |
| | | | 22 | 0.51 | 3.15 |
| Polynomial Curve fit of Results | | | 23 | 0.43 | 2.67 |
| Comparison | | Calads | 24 | 0.36 | 2.27 |
| Polynomial Coefficients | | Polynomial Coefficients | 25 | 0.30 | 1.94 |
| 1.612E+00 | | 1.595E+00 | 26 | 0.24 | 1.67 |
| −3.921E−02 | | −2.761E−02 | 27 | 0.20 | 1.45 |
| 1.436E−03 | | −6.960E−04 | 28 | 0.16 | 1.27 |
| −1.582E−04 | | −1.526E−05 | 29 | 0.12 | 1.13 |
| 3.150E−06 | | | 30 | 0.09 | 1.02 |
| | R2 = 9.9817E−01 | R2 = 9.9811E−01 | | | |

| | |
|---|---|
| Compare Poly | y = 3.1495E−06 × 4 − 1.5820E−04 × 3 + 1.4363E−03 × 2 − 3.9212E−02x + 1.6124E+00 |
| Calads Poly. | y = −1.5259E−05 × 3 − 6.9597E−04 × 2 − 2.7609E−02x + 1.5954E+00 |

Calculated Trace Capacity Numbers
Trace capacity no. Gas-phase TCN-G(g/100 cc) = 4.40
Acetoxime Trace capacity no. TCN(mg/cc) = 11.79
Mid capacity no. MCN(g/100 cc) = 5.91

GAED Raw Data

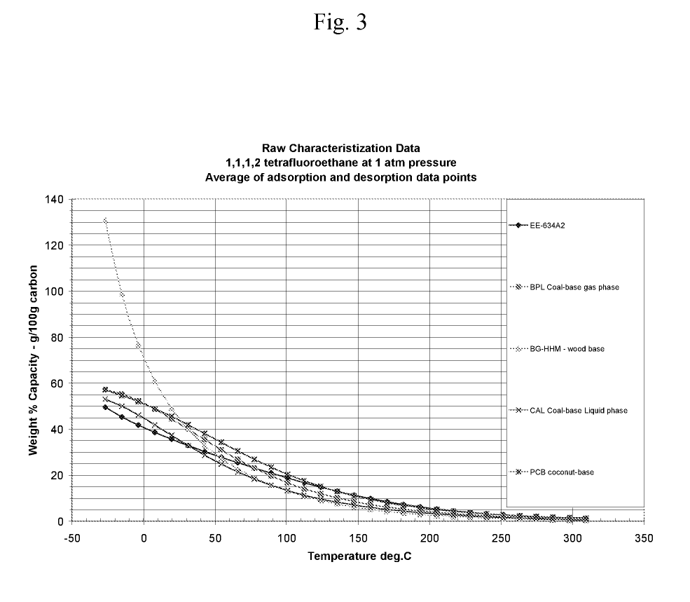

The GAED (gravimetric adsorption energy distribution method) measured over 400 adsorption and desorption data points covering seven orders of magnitude in relative pressure (isothermal basis) and three orders of magnitude in carbon loading. The mass adsorbed was also divided by the carbon mass to generate a weight percent loading for easier comparison. The raw data was plotted in FIG. 3. At 240° C., the adsorbent gas C134a or 1,1,1,2-tetrafluoroethane was introduced and the loading increased. In FIG. 3, it should be noted that the mass loading was plotted against temperature but the relative pressure was also changing. There were three variables affecting performance that changed from point to point: vapor pressure, partial pressure, and temperature.

To make comparisons easier, the large data file of adsorption/desorption points at different temperatures and relative pressures was simplified. First the data was interpolated to get 30 evenly spaced points covering the entire data range. Next the adsorption and desorption results were averaged to get the equilibrium values (the difference between adsorption and desorption was minimal for this sample—no hysteresis). The y-axis was converted to pore volume measures, in cc liquid adsorbed or cc pores filled/100 granms carbon, instead of weight percent. The average interpolated data for these characteristic curves is presented in Table 1, and FIGS. 2, 4A and 4B.

Performance Prediction Models

These curves were the only carbon related information required to predict physical adsorption performance using Polanyi Adsorption Potential theory. These single and multicomponent, gas and liquid phase, computer models were used to predict carbon performance and are available from PACS. To do performance predictions the following polynomial describes these carbon samples:

| Carbon name | Characteristic curve polynomial - 3rd degree |
|---|---|
| EE-634A2 | y = −1.5259E−05 × 3 − 6.9597E−04 × 2 − |
| | 2.7609E−02x + 1.5954E+00 |
| BPL Coal-base | y = 5.8955E−05 × 3 − 2.8880E−03 × 2 − |
| gas phase | 2.6182E−02x + 1.7029E+00 |
| BG-HHM - | y = −6.3875E−05 × 3 + 2.5948E−03 × 2 − |
| wood base | 1.1114E−01x + 2.0183E+00 |
| CAL Coal-base | y = 3.5299E−05 × 3 − 1.8375E−03 × 2 − |
| Liquid phase | 4.0325E−02x + 1.6682E+00 |
| PCB coconut-base | y = 5.6334E−05 × 3 − 3.0968E−03 × 2 − |
| | 1.3312E−02x + 1.6731E+00 |

In the equation, y was the common logarithm of pore volume in cc/100 g carbon and x was the e/4.6V adsorption potential in cal/cc.

Performance in the Six Types of Applications

The simplest comparison of carbon for a specific application was to run the performance prediction calculations for specific conditions, concentrations, and components present in the application. All physical adsorption applications can be placed into six application types. The comparative results in Table 2a and Table 2b demonstrate the value of the different carbons for use in the different types of applications on a volume basis. For a given application type, the results are related to the amount of carbon required to get a certain level of performance. Therefore, a carbon with twice the cc/100 g adsorption performance in an application type required half the pounds of carbon to achieve a level of performance in that application type.

Table 2a compares performance on a volume basis and weight basis respectfully, and gives the values of the comparative results for the sample carbons versus the performance for the standard commercial carbons for the six application types.

TABLE 2a
Performance in the Six Application Types on a Volume Basis

| | Carbon | | | | |
|---|---|---|---|---|---|
| | | BPL | | CAL | |
| | | Coal- | | Coal- | |
| | | base | BG-HHM - | base | PCB |
| | | gas | wood | Liquid | coconut- |
| | EE-634A2 | phase | base | phase | base |
| | Performance - Volume Basis | | | | |
| Application | cc/ | cc/ | cc/ | cc/ | cc/ |
| Type | 100 cc | 100 cc | 100 cc | 100 cc | 100 cc |
| Type I | 3.25 | 8.64 | 10.24 | 8.46 | 6.31 |
| Type II | 7.97 | 19.43 | 8.61 | 15.27 | 17.42 |
| Type III | 5.83 | 12.35 | 4.14 | 9.18 | 12.17 |
| Type IV | 1.65 | 2.88 | 0.91 | 2.10 | 3.09 |
| Type V | 1.60 | 2.22 | 0.59 | 1.73 | 2.58 |
| Type VI | 0.51 | 0.78 | 0.16 | 0.52 | 0.89 |

TABLE 2b
Performance in the Six Application Types on a Weight Basis

| | Carbon | | | | |
|---|---|---|---|---|---|
| | | BPL | | CAL | |
| | | Coal- | | Coal- | |
| | | base | BG-HHM - | base | PCB |
| | | gas | wood | Liquid | coconut- |
| | EE-634A2 | phase | base | phase | base |
| Application | Performance - Weight Basis | | | | |
| Type | g/100 g | g/100 g | g/100 g | g/100 g | g/100 g |
| Type I | 14.81 | 20.23 | 61.84 | 21.28 | 16.78 |
| Type II | 36.32 | 45.48 | 52.02 | 38.44 | 46.34 |
| Type III | 26.58 | 28.91 | 25.00 | 23.10 | 32.38 |
| Type IV | 7.51 | 6.74 | 5.48 | 5.30 | 8.22 |
| Type V | 7.28 | 5.19 | 3.54 | 4.36 | 6.86 |
| Type VI | 2.35 | 1.82 | 0.94 | 1.32 | 2.36 |

Type I Regenerable Heavy Loading Applications

Type II Heavy Loading Applications

Type III Moderate Loading Applications

Type IV Regenerable Trace Loading Applications

Type V Trace Loading Applications

Type VI Ultra Trace Loading Applications

Adsorption Isotherms

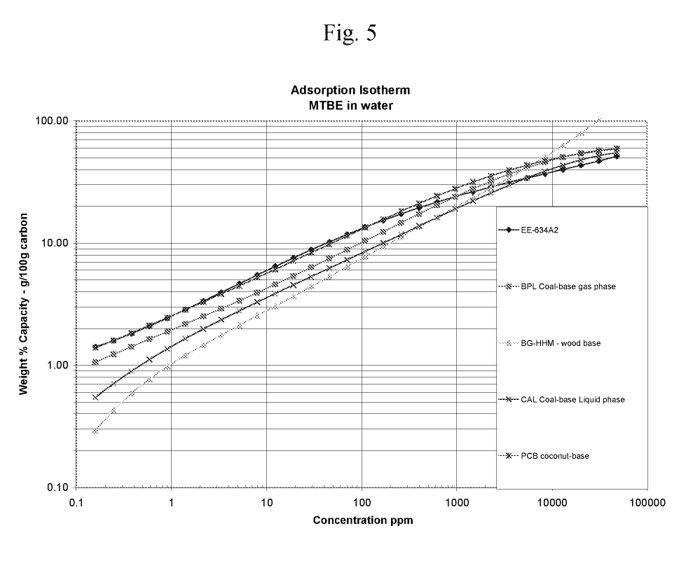

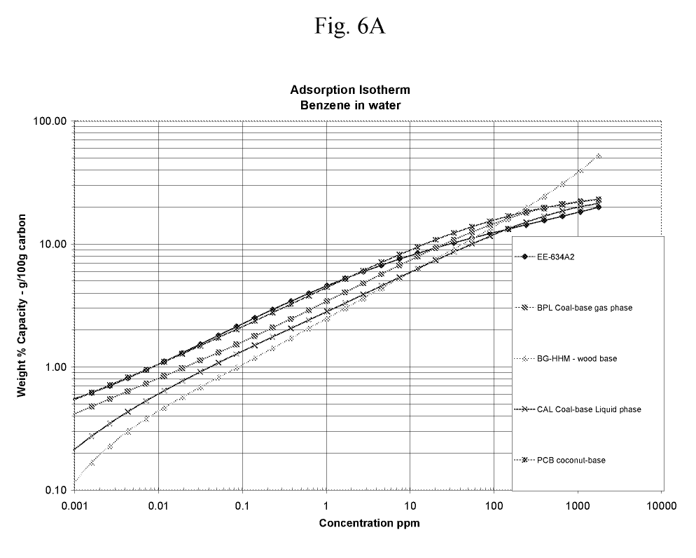

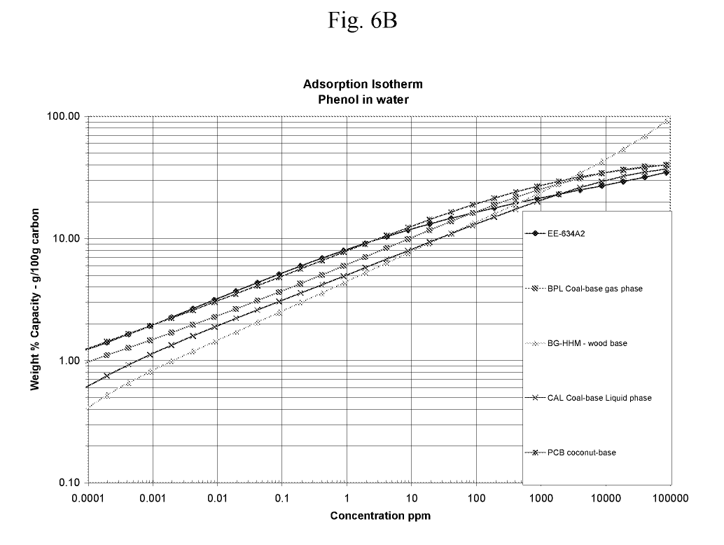

The characteristic curves are also translated into adsorption isotherms using the programs mentioned above: FIG. 5 for MTBE (weakly adsorbed material), FIG. 6A for benzene (more strongly adsorbed species) and FIG. 6B for phenol at pH 7 (quite strongly adsorbed material). These graphs of the calculated aqueous-phase isotherms, showed this sample had very good trace capacity activity as compared to the other reference samples for MTBE, Benzene and Phenol.

Pore Size Distributions

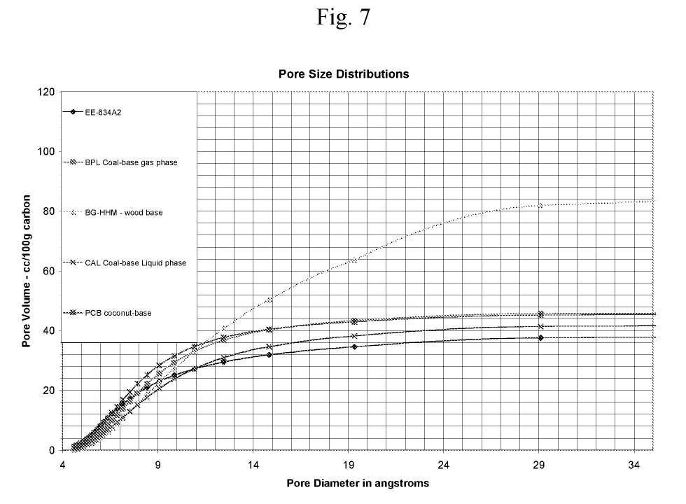

The Kelvin equation, modified by Halsey, can be used to convert the characteristic curve data to calculated BET surface areas or pore size distributions. This is not useful in terms of performance evaluations, but some audiences are more comfortable with the concepts of pore radius and a series of capillary sizes when thinking about activated carbon. FIG. 7 shows the cumulative pore size distributions. The single and multi-point BET surface areas were calculated from these curves and are presented in Table 1.

Application performance tests show how this material would perform with the performance prediction calculations for specific applications. The Type IV (Regenerable Trace Loading Applications like Acetone Solvent Recovery), Type V (Trace Loading Applications like Trichloroethane from Water) and Type VI (Ultra Trace Loading Applications like Vinyl Chloride from Water) were this carbon's areas of best performance. The conditioned sample had about 93% of the total adsorption pore volume as the CAL Coal-based Liquid phase reference material (Table 3). The calculated BET surface area indicated that this GAC had a calculated surface area of 703 sq.meters/g, about 80% of the PCB Reference material (Table 1). The Differential Characteristic Curves in FIG. 4a showed the structure of this material to be more like that of BG-HHM wood-based reference and also had increased pore structure at the larger pore area.

TABLE 3
Carbon Characteristic Curves - Cumulative basis
ADSORPTION POTENTIAL DISTRIBUTIONS
Carbon Pore Volume Data 10/06 CDM

| Contour | EE-634A2 | BPL Coal-base gas phase | BG-HHM - wood base | CAL Coal-base Liquid phase | PCB coconut-base |
|---|---|---|---|---|---|
| Line Number | My Act (EE-634A2) | BPL Coal-base gas phase | BG-HHM Wood-base | CAL Coal-base Liquid phase | PCB Std |
| or | 42248.00 | 38082.00 | 38258.00 | 38035.00 | 38101.00 |
| Adsorption | Auto GAED ver. 10/09 | Auto GAED ver. 10/09 | Auto Apr. 10, 2004 Prgm | Auto 2004 Ramp Prgm | Auto GAED ver. 10/06 |
| Potential | Capacity | Capacity | Capacity | Capacity | Capacity |
| e/4.6 V | cc/100 g · C. | cc/100 g · C. | cc/100 g · C. | cc/100 g · C. | cc/100 g · C. |
| 0 | 40.96 | 47.35 | 108.17 | 43.87 | 47.25 |
| 1 | 37.54 | 45.74 | 81.89 | 41.37 | 45.20 |
| 2 | 34.55 | 43.32 | 63.58 | 38.18 | 42.96 |
| 3 | 31.89 | 40.26 | 50.40 | 34.61 | 40.44 |
| 4 | 29.46 | 36.78 | 40.62 | 30.89 | 37.67 |
| 5 | 27.19 | 33.06 | 33.14 | 27.23 | 34.68 |
| 6 | 25.05 | 29.30 | 27.29 | 23.76 | 31.55 |
| 7 | 22.99 | 25.65 | 22.60 | 20.56 | 28.37 |
| 8 | 21.01 | 22.22 | 18.78 | 17.68 | 25.24 |
| 9 | 19.11 | 19.08 | 15.63 | 15.14 | 22.24 |
| 10 | 17.27 | 16.28 | 13.02 | 12.93 | 19.43 |
| 11 | 15.50 | 13.82 | 10.83 | 11.02 | 16.86 |
| 12 | 13.82 | 11.69 | 8.99 | 9.38 | 14.54 |
| 13 | 12.24 | 9.87 | 7.46 | 7.99 | 12.49 |
| 14 | 10.76 | 8.33 | 6.19 | 6.81 | 10.69 |
| 15 | 9.40 | 7.04 | 5.13 | 5.80 | 9.13 |
| 16 | 8.15 | 5.95 | 4.25 | 4.95 | 7.80 |
| 17 | 7.03 | 5.05 | 3.53 | 4.23 | 6.65 |
| 18 | 6.02 | 4.29 | 2.93 | 3.61 | 5.68 |
| 19 | 5.14 | 3.66 | 2.44 | 3.08 | 4.86 |
| 20 | 4.38 | 3.14 | 2.03 | 2.62 | 4.16 |
| 21 | 3.71 | 2.70 | 1.70 | 2.22 | 3.56 |
| 22 | 3.15 | 2.32 | 1.41 | 1.88 | 3.06 |
| 23 | 2.67 | 2.01 | 1.17 | 1.58 | 2.63 |
| 24 | 2.27 | 1.74 | 0.96 | 1.32 | 2.27 |
| 25 | 1.94 | 1.51 | 0.78 | 1.09 | 1.95 |
| 26 | 1.67 | 1.31 | 0.62 | 0.89 | 1.69 |
| 27 | 1.45 | 1.13 | 0.47 | 0.71 | 1.46 |
| 28 | 1.27 | 0.98 | 0.34 | 0.56 | 1.27 |
| 29 | 1.13 | 0.85 | 0.23 | 0.44 | 1.11 |
| Density g/cc | 0.265 | 0.516 | 0.200 | 0.480 | 0.454 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An activated carbon prepared from lignin residues of a pretreated biomass wherein the lignin residues comprise at least 50% of solid particles from about 5 microns to about 150 microns in size.

2. The activated carbon of claim 1, wherein a dry ash content of the activated carbon is below 3.0%.

3. The activated carbon of claim 1, wherein a dry oxygen content of the activated carbon is above 9%.

4. The activated carbon of claim 1, wherein a dry sulfur content of the activated carbon is below 0.40%.

5. The activated carbon of claim 1, wherein the activated carbon is powdered activated carbon (PAC), granular activated carbon (GAC), pelleted activated carbon, (EAC), extruder activated carbon, bead activated carbon (BAC), graphite, impregnated carbon or a combination thereof.

6. The activated carbon of claim 1, wherein the activated carbon has a particle size of: from about 5 microns to about 40 microns, about 5 microns to about 60 microns, about 5 microns to about 180 microns, less than about 100 microns, less than about 200 microns, less than about 170 microns, or less than about 250 microns.

7. The activated carbon of claim 1, wherein the activated carbon is produced from lignin residues having a particle size ranging from about 5 microns to about 200 microns.

8. A method of preparing an activated carbon, comprising:
(a) Loading a biomass into a pretreatment system comprising a reaction compartment;
(b) Pretreating the biomass within the reaction compartment with an elevated temperature, a substantially constant temperature, an acid, and steam for a period of time to produce a pretreated material;
(c) Adding water and neutralizing the resultant solubilized pretreated material;
(d) Hydrolyzing the solubilized pretreated material to produce a sugar-rich hydrolyzate product and lignin solids;
(e) Separating the lignin solids from the sugar rich hydrolyzate product; and
(f) Drying, carbonizing, and activating the lignin solids to produce an activated carbon.

9. The method of claim 8, wherein the hydrolyzing is done by enzymes and/or a biocatalyst.

10. The method of claim 8, wherein the activated carbon is powdered activated carbon (PAC), granular activated carbon (GAC), pelleted activated carbon, (EAC), extruder activated carbon, (BAC) bead activated carbon, or a combination thereof.

11. The method of claim 8, wherein the activated carbon has a particle size of: from about 5 microns to about 40 microns, from about 5 microns to about 60 microns, from about 5 microns to about 180 microns, less than about 100 microns, less than about 200 microns, less than about 170 microns, or less than about 250 microns.

12. (canceled)

13. The method of claim 8, wherein separating the lignin solids is done by flocculation, filtration, centrifugation, or a combination of one or more of these.

14. The method of claim 8, wherein at least 50% of the solid particles in the pretreated biomass composition are from about 5 micron to about 150 micron in size.

15. The method of claim 8, wherein the carbonizing occurs for a time period of: about 30 sec to about 1 min, about 1 min to about 5 min, about 5 min to about 1 hour, about 1 hour to about 24 hours, about 1 hour to about 18 hours, about 1 hour to about 12 hours, about 1 hour to about 6 hours, about 2 hours, about 3 hours, about 4 hours, about 6 hours, about 7 hours, about 8 hours, about 9 hours, about 10 hours, about 11 hours, about 13 hours, about 14 hours, about 15 hours, about 17 hours, about 19 hours, about 20 hours, about 21 hours, about 22 hours, or about 23 hours.

16. The method of claim 8, wherein the activating is done through physical or chemical activation.

17. The method of claim 8, wherein the activated carbon is impregnated activated carbon.

18. The method of claim 8, wherein the carbonization is conducted at a temperature of: about 150° C. to about 300° C., about 150° C. to about 250° C., about 150° C. to about 200° C., about 160° C., about 170° C., about 180° C., about 190° C., about 210° C., about 220° C., about 230° C., about 240° C., about 260° C., about 270° C., about 280° C., or about 290° C., or about 300° C., or about 350° C., or about 400° C., or about 450° C., or about 500° C., or about 550° C., or about 600° C., or about 650° C.

19. The method of claim 8, wherein the activation uses ZnCl₂, H₃PO₄, Na₂CO₃, K₂CO₃, KOH, CO₂, or alkali metal compounds.

20. (canceled)

21. (canceled)

22. (canceled)

23. (canceled)

24. A system for producing activated carbon from a biomass comprising cellulose, hemicellulose, and lignocellulose, the system comprising:
(a) A pretreatment unit to pretreat the biomass to produce a pretreated biomass composition comprising solid particles and a mixture of C5 and C6 polymers, monomers and dimers by:
(i) hydration of the biomass composition in a non-neutral pH aqueous medium to produce a hydrated biomass composition,
(ii) mechanical size reduction of the hydrated biomass composition to produce the solid particles, and
(iii) heating the hydrated biomass composition under pressure for a time sufficient to produce the pretreated biomass composition comprising solid particles and a mixture of C5 and C6 polymers, monomers and/or dimers;
(b) An enzymatic hydrolysis unit to produce a sugar stream and lignin residues from the pretreated biomass composition by:
(i) neutralizing the pretreated biomass composition to produce a pH aqueous medium close to neutral; and
(ii) contacting the pretreated biomass composition with one or more enzymes for a time sufficient to produce lignin residues and a sugar stream;
(c) A separation unit to separate the sugar stream from the lignin residues;
(d) A carbonization unit to carbonize the lignin residues to produce charcoal from the lignocellulose residues; and
(e) An activation unit to activate the charcoal to produce an activated carbon.

25.-36. (canceled)

37. The system of claim 24, wherein the biomass composition comprises alfalfa, algae, bagasse, bamboo, sorghum corn stover, corn fiber, corn cobs, corn kernels, corn mash, corn steep liquor, corn steep solids, distiller's grains, distiller's dried solubles, distiller's dried grains, condensed distiller's solubles, distiller's wet grains, distiller's dried grains with solubles, eucalyptus, food waste, fruit peels, garden residue, grass, grain hulls, modified crop plants, municipal waste, oat hulls, coconut shells, nuts, nut shells, paper, paper pulp, prairie bluestem, poplar, rice hulls, seed hulls, silage, sorghum, straw, sugarcane, switchgrass, wheat, wheat straw, wheat bran, de-starched wheat bran, willows, wood, sawdust, wood chips, plant cells, plant tissue cultures, tissue cultures, or a combination thereof.

38.-49. (canceled)
